(19) United States
(12) Patent Application Publication
(10) Pub. No.: US 2025/0367461 A1
(43) Pub. Date: Dec. 4, 2025

(54) SMART GARMENT

(71) Applicant: Myant Inc., Etobicoke (CA)

(72) Inventors: Ladan Eskandarian, Toronto (CA); Milad Alizadeh-Meghrazi, Toronto (CA); Tony Chahine, Toronto (CA); Brian M. Etzel, Butler, PA (US); Saundra L. Penkunas, Portland, OR (US); Christopher L. Swenglish, Connellsville, PA (US)

(21) Appl. No.: 19/235,483

(22) Filed: Jun. 11, 2025

Related U.S. Application Data
(63) Continuation of application No. PCT/US2023/083925, filed on Dec. 13, 2023.
(60) Provisional application No. 63/432,477, filed on Dec. 14, 2022.

(51) Int. Cl. A61N 1/39; A41D 1/00; D04B 1/16; D04B 1/24; D06M 15/63; D06M 15/71; D06M 101/30; D06M 101/34

(52) U.S. Cl. CPC A61N 1/3904; A41D 1/005; A61N 1/3987; D04B 1/16; D04B 1/24; D06M 15/63; D06M 15/71; D06M 2101/30; D06M 2101/34; D06M 2200/00; D10B 2331/02; D10B 2331/14; D10B 2509/00

(57) ABSTRACT

A non-invasive, wearable, ambulatory device capable of cardiac defibrillation includes a smart garment to be worn by a patient. The device also includes a plurality of therapeutic electrodes configured to be removably attached to the garment. A plurality of polymer-based ECG sensing electrodes are configured to provide ECG signals based on skin electrical activity of the patient wearing the smart garment. Polymer-based ECG sensing electrodes are formed by applying a conductive polymer fluid to each of a plurality of base fibers to form a plurality of individually conductive polymer coated fibers. The base fibers are single fibers and/or multi-fibers. The plurality of individually conductive polymer coated fibers are assembled into the one or more plurality of polymer-based ECG sensing electrodes. A controller is configured to receive the ECG signals, determine at least one arrhythmia episode based on the received ECG signals, and to cause a defibrillation shock.

### CROSS REFERENCE TO RELATED APPLICATIONS

This patent application is a continuation of PCT patent application no. PCT/US2023/083925, filed Dec. 13, 2023, and entitled “SMART GARMENT,” which in turn claims priority from U.S. provisional patent application No. 63/432,477, filed on Dec. 14, 2022, the disclosures of which are each incorporated herein, in their entirety, by reference.

### FIELD IN THIS DISCLOSURE

Illustrative embodiments in this disclosure generally relate to smart garments, including smart garments for physiological monitoring.

### BACKGROUND

Sensory devices, such as physiological data sensors, may be integrated or embedded into garments. As an example, smart garments may be used for medical applications, such as for wearable cardioverter defibrillators. Smart garments may also be used to help with monitoring and improving athletic performance. When sensory devices are embedded into garments, the sensory devices may be positioned physically proximate to user limbs or body parts. The garments having the sensory devices embedded therein may be worn by users for extended durations of time. ECG electrodes are used to sense cardiac activity in a user.

### SUMMARY

In accordance with an embodiment, a non-invasive, wearable, ambulatory device capable of cardiac defibrillation includes a smart garment configured to be worn about a torso of a patient. The device also includes a plurality of therapeutic electrodes configured to be removably attached to the garment. A plurality of polymer-based ECG sensing electrodes are configured to provide ECG signals based on skin electrical activity of the patient wearing the smart garment. One or more plurality of polymer-based ECG sensing electrodes is formed by applying a conductive polymer fluid to each of a plurality of base fibers to form a plurality of individually conductive polymer coated fibers. The base fibers are single fibers and/or multi-fibers. The polymer-based ECG sensing electrode is also formed by assembling the plurality of individually conductive polymer coated fibers into the one or more plurality of polymer-based ECG sensing electrodes of the smart garment. The device also includes a controller in electrical communication with the plurality of therapeutic electrodes and the plurality of polymer-based ECG sensing electrodes. The controller is configured to receive the ECG signals and determine at least one arrhythmia episode occurring in the patient based on the received ECG signals. The controller is further configured to cause a defibrillation shock to be delivered to the patient via the plurality of therapeutic electrodes as a function of determining the occurrence of the at least one arrhythmia episode. In one or more examples, a smart garment may comprise a garment having: one or more electrodes attached to or otherwise incorporated therein for contacting the wearer; one or more sensors attached to or incorporated therein for obtaining data from the wearer; one or more processors attached to or incorporated therein for processing information about the wearer of the garment; and/or one or more power sources attached to or incorporated therein for powering the one or more sensors, if present, and one or more processors, if present. In one or more examples, a smart garment may comprise a garment incorporating one or more textiles that facilitates the integration of electronic components (e.g., electrodes, sensors, and/or processors) into the garment.

In various embodiments, assembling the plurality of individually conductive polymer coated fibers includes weaving the plurality of individually conductive polymer coated. Additionally, or alternatively, assembling the plurality of individually conductive polymer coated fibers may include knitting the plurality of individually conductive polymer coated. In various embodiments, a stretchable fabric portion of the smart garment at least partially surrounds the polymer-based ECG sensing electrodes. A yield strain ratio of the stretchable fabric portion relative to the polymer-based ECG sensing electrodes may range between about 1.1 to about 6.0.

Among other things, the conductive polymer fluid may include poly(3,4-ethylenedioxythiophene) polystyrene sulfonate (PEDOT:PSS). The conductive polymer fluid may also include ethylene glycol. The conductive polymer fluid may have a surface tension of between about 30 mN/m and about 45 mN/m, or about 35 mN/m and about 40 mN/m, or about 39 mN/m. The conductive polymer fluid may have a viscosity of between about 65 Centipoise and about 85 Centipoise.

In various embodiments, one or more polymer-based ECG sensing electrodes are configured to be removably attached to the smart garment. The one or more polymer-based ECG sensing electrodes may be configured to be removably attached to the smart garment by one or more of: hook and loop fasteners, snap connectors, and/or adhesive material. Two or more of the plurality of polymer-based ECG sensing electrodes may be electrically coupled by a polymer fiber interconnect. The polymer fiber interconnect may be formed by assembling the plurality of the individually conductive polymer coated fibers in a longitudinal pattern between two of the plurality of polymer-based ECG sensing electrodes. The interconnect may form an exposed top layer. Additionally, or alternatively, the interconnect may be positioned beneath a first non-conductive fabric.

The plurality of polymer-based ECG sensing electrodes may be positioned over a first non-conductive fabric. The base fibers may include yarn, nylon 6 and/or nylon 6,6. A surface of the base fiber may be pre-treated with plasma prior to applying the conductive fluid. The surface of the base fiber may be pre-treated with the plasma between about 1 hour to about 72 hours, or between about 6 hours and about 48 hours, or between about 12 hours and about 24 hours, or about 24 hours, prior to applying a conductive polymer fluid. The non-conductive fabric fiber may include a round, hollow round, triangle, hollow triangle, trilobal, hollow trilobal, square, hollow square, scalloped oval, hexachannel, cruciform, flat, rectangular, and/or arrow cross-sectional shape.

In various embodiments, applying a conductive polymer fluid includes passing the fiber through squeeze rolls and curing the conductive polymer fluid at between about 160 C and about 220 C. A coating rate of the applying the conductive polymer fluid to the fibers may be between about 50 uL/min and 250 uL/min. A coating speed of the applying the conductive polymer fluid to the fibers may be between about 10 rpm and about 40 rpm.

The polymer-based ECG sensing electrodes may each have a signal-to-noise ratio of between 2.5 and 30.1 for the received ECG signals. The polymer-based ECG sensing electrodes may each have a skin-electrode impedance value of between 65 kOhms and 105 kOhms at 100 Hz A resistance of the polymer-based ECG sensing electrodes may change less than a predetermined 50% of a baseline impedance value from 10 Hz to about 500 Hz after 30 wash cycles. Indeed, the polymer-based ECG sensing electrodes impedance may change less than a predetermined 75% of a baseline impedance value after 60 wash cycles.

In accordance with another embodiment, a non-invasive, wearable, ambulatory device capable of cardiac defibrillation includes a smart garment configured to be worn about a torso of a patient. The device includes a plurality of therapeutic electrodes configured to be removably attached to the garment. The device also includes a plurality of polymer-based ECG sensing electrodes configured to provide ECG signals based on skin electrical activity of the patient wearing the smart garment. One or more plurality of polymer-based ECG sensing electrodes includes a plurality of individually conductive polymer coated fibers. Each of the plurality of the individually conductive polymer coated fibers may include a base fiber treated with a conductive polymer fluid disposed along the base fiber. The base fiber may be a single fiber and/or multifiber. The plurality of the individually conductive polymer coated fibers may be arranged in a predetermined configuration. The device includes a controller in electrical communication with the plurality of therapeutic electrodes and the plurality of polymer-based ECG sensing electrodes. The controller is configured to receive the ECG signals, and determine at least one arrhythmia episode occurring in the patient based on the received ECG signals. The controller may further be configured to cause a defibrillation shock to be delivered to the patient via the plurality of therapeutic electrodes as a function of determining the occurrence of the at least one arrhythmia episode.

In various embodiments, the conductive polymer fluid may form a coating on the base fiber. The base fiber may be a non-conductive fiber.

In accordance with yet another embodiment, a non-invasive, wearable, ambulatory device capable of cardiac defibrillation includes a plurality of therapy electrodes configured to deliver one or more defibrillation pulses to the patient. The device also includes a smart garment configured to be worn around a torso of the patient. The smart garment includes a stretchable fabric portion having a first yield strain value. The smart garment also includes a plurality of biopotential recording fabric portions. Each of the biopotential recording fabric portions is formed from a plurality of assembled individually conductive polymer coated fibers. The plurality of biopotential recording fabric portions are configured to sense ECG signals from a patient. The plurality of biopotential recording fabric portions have a second yield strain value that is less than the first yield strain value.

In various embodiments, the stretchable fabric portion at least partially surrounds the plurality of biopotential recording fabric portions such that the smart garment is configured to maintain continuous electrical contact between the plurality of biopotential recording fabric portions and skin of the patient over a duration of time when the smart garment is worn about the torso of the patient. The individually conductive polymer coated fibers may be coated with PEDOT:PSS.

In various embodiments, the stretchable fabric portion may surround the biopotential recording fabric portion circumferentially. The stretchable fabric portion may be layered underneath of the biopotential recording fabric portion. The individually conductive fibers may be weaved together to form the biopotential recording fabric portion. The individually conductive fibers may be formed from nylon.

In accordance with yet another embodiment, a method of making a smart garment for cardiac health monitoring individually coats each of a plurality of single fibers and/or multifibers with a conductive polymer coating fluid to form a plurality of conductive fabric fibers. The method assembles the plurality of conductive fabric fibers to form an electrically conductive fabric portion of a smart garment. The electrically conductive fabric portion forms an ECG electrode configured to sense ECG signals from a patient. A stretchable fabric portion of the smart garment at least partially surrounding the electrically conductive fabric portion.

The stretchable fabric portion may has a first yield strain value, and the electrically conductive fabric portion has a second yield strain value that is less than the first yield strain value. Alternatively, the first yield strain value may be less than the second yield strain value. The method may assemble portions of the smart garment (e.g., the conductive fabric portion) by knitting, weaving, or embroidering. In various embodiments, the ECG electrode may be knitted using a Stoll CMS-ADF flatbed knitting machine.

The method may cure the plurality of conductive fabric fibers before assembling the plurality of conductive fabric fibers. Curing may include continuously moving the fibers through an oven. Curing may further include heating the fibers at a temperature of between about 190 C and 220 C.

Various embodiments use a coating speed of between 10 rpm and 40 rpm. Furthermore, the coating may be deposited on the fiber at a rate of between 50 uL/min and 150 uL/min. The linear density of the coating may be between 20 uL/m and 35 uL/m.

Among other shapes, the fiber may have a ribbon, trilobal, or circular cross-section cross-section. The fiber may comprise nylon, carbon, and/or polyester. The conductive polymer coating fluid may include PEDOT:PSS. The conductive polymer coating fluid may have a viscosity between about 70 cps and about 75 cps. The conductive polymer coating fluid may have a surface tension between about 35 mN/m and about 45 mN/m.

The method may also knit a plurality of conductive fabric fibers to form a plurality of ECG electrodes. A plurality of conductive fabric fibers may be knitted to form a plurality of interconnects extending between the plurality of ECG electrodes that electrically couple the plurality of ECG electrodes.

Various embodiments may plasma treat a surface of the fiber prior to coating the non-conductive fabric fiber. Coating may occur within 24 hours after the fiber is plasma treated. Coating may include passing the fabric through squeeze rolls. In various embodiments, the ECG electrode may have an impedance value of less than 100 kOhms at 100 Hz. The ECG electrode resistance may change less than 50% after 30 wash cycles.

A non-invasive, wearable, ambulatory device capable of cardiac monitoring, the device comprising: a smart garment configured to be worn about a torso of a patient; a plurality of polymer-based ECG sensing electrodes configured to provide ECG signals based on skin electrical activity of the patient wearing the smart garment, wherein one or more plurality of polymer-based ECG sensing electrodes is formed by: applying a conductive polymer fluid to each of a plurality of base fibers, the base fibers being single fibers and/or multi-fibers, to form a plurality of individually conductive polymer coated fibers, and assembling the plurality of individually conductive polymer coated fibers into the one or more plurality of polymer-based ECG sensing electrodes of the smart garment; and a controller in electrical communication with the plurality of therapeutic electrodes and the plurality of polymer-based ECG sensing electrodes, the controller configured to receive the ECG signals and provide an output based on said received ECG signals.

Illustrative embodiments in this disclosure are implemented as a computer program product having a computer usable medium with computer readable program code thereon. The computer readable code may be read and utilized by a computer system in accordance with conventional processes.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification, but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIG. 1 schematically shows a user wearing a smart garment in accordance with illustrative embodiments in this disclosure.

FIG. 2A schematically shows medical devices that may be coupled to and/or integrated with the smart garment in accordance with illustrative embodiments in this disclosure.

FIG. 2B schematically shows an alternative medical device that may be coupled to and/or integrated with the smart garment in accordance with illustrative embodiments in this disclosure.

FIG. 3A schematically shows a patient wearing the smart garment having the medical devices in accordance with illustrative embodiments in this disclosure.

FIG. 3B schematically shows an alternative of the smart garment having the medical devices in accordance with illustrative embodiments in this disclosure.

FIG. 4A schematically shows an example smart garment in accordance with illustrative embodiments in this disclosure.

FIG. 4B schematically shows a knit assembly of the fibers of the smart garment in accordance with illustrative embodiments in this disclosure.

FIG. 4C schematically shows a weave assembly of the fibers of the smart garment in accordance with illustrative embodiments in this disclosure.

FIG. 4D schematically shows a magnified view of a portion of the smart garment of FIG. 4A.

FIG. 4E schematically sows a cross-section of FIG. 4D.

FIG. 4F schematically shows a magnified view of another portion of the smart garment of FIG. 4A.

FIG. 4G schematically shows a cross-sectional view of FIG. 4F.

FIG. 5 shows a process of making and using the smart garment in accordance with illustrative embodiments.

FIG. 6A schematically shows a variety of cross-sectional fiber shapes in accordance with illustrative embodiments.

FIG. 6B schematically shows plasma treating the base fiber in accordance with illustrative embodiments.

FIG. 7A schematically shows a roll-to-roll coating process for applying a conductive polymer fluid to the base fiber in accordance with illustrative embodiments.

FIG. 7B schematically shows a cross-section of a coated base fiber in accordance with illustrative embodiments.

FIG. 7C schematically shows an alternative process for applying a conductive polymer fluid to the base fiber in accordance with illustrative embodiments.

FIG. 8A schematically shows a plurality of individually conductive polymer coated fibers assembled after coating.

FIG. 8B schematically shows the plurality of individually conductive polymer coated fibers of FIG. 8A in contact with the patient.

FIG. 8C schematically shows a plurality of coated conductive fibers assembled before coating.

FIG. 8D schematically shows the plurality of coated conductive fibers of FIG. 8C in contact with the patient.

FIGS. 9A-9B schematically show a removable ECG electrode in accordance with illustrative embodiments.

FIG. 9C schematically shows another embodiment of a removable ECG electrode in accordance with illustrative embodiments.

FIG. 9D schematically shows another example of a removable ECG electrode in accordance with illustrative embodiments.

FIG. 10A schematically shows polymer-based ECG sensing electrodes disposed on the sides of the patient's body and on an anterior and posterior position of the patient's body in accordance with illustrative embodiments.

FIGS. 10B-10C schematically show the polymer-based ECG sensing electrodes disposed in the smart garment in standard locations in accordance with illustrative embodiments.

FIGS. 11A-11E shows the through-plane impedance amplitude of unwashed, 30× washed, and 60× washed fabrics made of PEDOT:PSS-coated fibers at frequency of 10 Hz in accordance with illustrative embodiments. FIG. 11F schematically shows the average impedance amplitude for the fibers of FIGS. 11A-11E at a variety of different frequencies in accordance with illustrative embodiments.

FIG. 12A shows a coating system used to create conductive fibers in accordance with illustrative embodiments.

FIG. 12B shows an assembling system used to assemble conductive fibers into a conductive fabric portion in accordance with illustrative embodiments.

FIG. 13 schematically shows a liquid drop showing the quantities of force balance at the interface in accordance with illustrative embodiments.

FIGS. 14A-14F show pictures of magnified cross-sections of PA 66 fibers in accordance with illustrative embodiments.

FIGS. 15A-15F schematically show surface plots of impedance values for unwashed and washed electrodes in accordance with illustrative embodiments.

FIGS. 16A-16E show average impedance amplitude of unwashed, 30× washed, and 60× washed electrodes at 1 Hz, 10 Hz, 100 Hz, and 1000 Hz in accordance with illustrative embodiments.

FIGS. 17A-17E shows Average electrode-skin impedance amplitude of unwashed, 30× washed, and 60× washed electrodes at 1 Hz, 10 Hz, 100 Hz, and 1000 Hz in accordance with illustrative embodiments.

FIG. 18A shows the placement of textile and gel electrodes on the subject in accordance with illustrative embodiments of the invention. FIGS. 18B-18F shows on-skin ECG measurements using unwashed, 30× washed, and 60× washed electrodes in accordance with illustrative embodiments.

FIG. 19 schematically shows a controller of the smart garment in accordance with illustrative embodiments in this disclosure.

### DETAILED DESCRIPTION

This disclosure relates to techniques, processes, and devices implementing electrodes/sensors comprising a plurality of conductive fibers. One example type of the electrodes/sensors are polymer-based ECG sensing electrodes that are configured to provide ECG signals based on skin electrical activity of a patient wearing a smart garment. To that end, the smart garment includes a plurality of conductive fibers formed by applying a conductive polymer fluid, such as poly(3,4-ethylenedioxythiophene) polystyrene sulfonate, to a plurality of base fibers, such as non-conductive yarns, to form conductive fibers. The conductive fibers are then assembled to form the polymer-based electrode/sensor, such as an ECG sensing electrode. Details of illustrative embodiments are discussed below. Various embodiments may refer to “polymer-based ECG sensing electrodes” and “polymer-based sensing electrodes” interchangeably in the following description.

Such polymer-based ECG sensing electrodes are advantageously more comfortable on the skin of subjects, users, or patients, when compared to conventional ECG electrodes. For example, in the context of continuous and/or long term ECG monitoring, polymer-based ECG sensing electrodes may be better tolerated than conventional ECG electrodes on human skin (e.g., when tested in accordance with ANSI/AAMI/ISO 10993-10:2010 standards for Biological Evaluation of Medical Devices—Part 10: Tests for Irritation and Skin Sensitization as described in further detail below). Polymer-based ECG sensing electrodes are more flexible and as such better able to conform to the contours of the patient's anatomy than conventional ECG electrodes that may be built from rigid metallic materials. Moreover, as described in more detail below, polymer-based ECG sensing electrodes are based on a fabric or yarn substrate. For at least these reasons, polymer-based ECG sensing electrodes as described herein are suited for a variety of applications involving close, intimate contact with human skin, including for use in continuous and/or long term sensing of cardiac activity for exercise monitoring and medical grade garments. In this regard, polymer-based ECG sensing electrodes can promote better patient or user compliance than where conventional ECG electrodes are used. As an example, polymer-based ECG sensing electrodes promote can promote continuous use or wear of garments or devices based on such electrodes, e.g., a patient removes or minimizes interruptions in use or wear. Additionally or alternatively, polymer-based ECG sensing electrodes promote can promote longer term use or wear of the garments or devices. In all such cases, overall patient or user compliance with the prescribed, intended, or designed use of the garment or devices is improved relative to conventional ECG electrodes. This improved overall patient or user compliance results in better quality ECG data for use in exercise monitoring, arrhythmia monitoring and treatment, or in reliable cardiac metric calculations derived from the ECG data. For example, as disclosed herein, polymer-based ECG sensing electrodes can be used in smart garments for non-invasive, wearable, ambulatory devices capable of cardiac defibrillation.

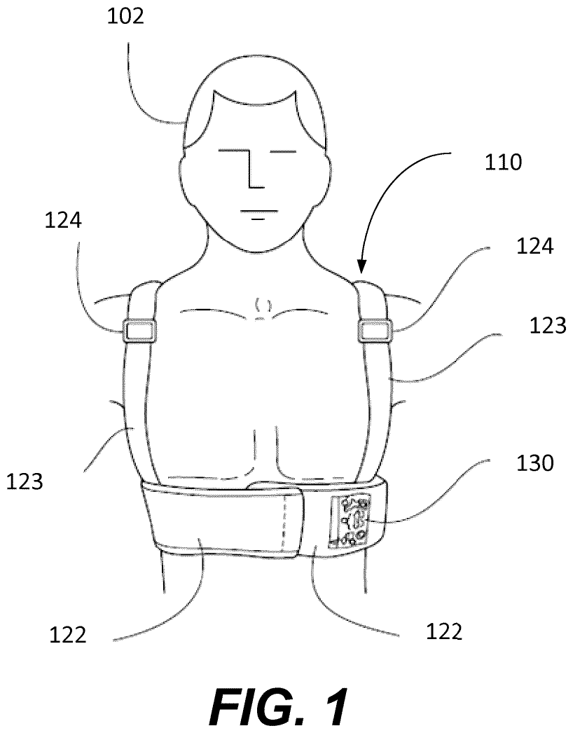

FIG. 1 schematically shows a user 102 (e.g., patient 102) wearing a smart garment 110 in accordance with illustrative embodiments in this disclosure. Among other uses, the smart garment 110 may include a wide variety of electronic and mechanical devices for monitoring and treating patients' 102 medical conditions such as cardiac arrhythmias including sudden cardiac arrest. In some examples, depending on the underlying medical condition being monitored or treated (e.g., ventricular tachycardia and/or ventricular fibrillation), devices such as cardiac defibrillators may be externally connected to the patient 102. In some cases, physicians may use devices alone or in combination with drug therapies to treat conditions such as cardiac arrhythmias.

The smart garment 110 may be provided in the form of a vest or harness having a back portion and sides extending around the front of the patient 102 to form a belt 122. The ends of the belt 122 are connected at the front of the patient 102 by a closure, which may comprise one or more clasps. Multiple corresponding closures may be provided along the length of the belt 122 to allow for adjustment in the size of the secured belt 122 in order to provide a more customized fit to the patient 102. The smart garment 110 may further include two straps 123 connecting the back portion to the belt 122 at the front of the patient 102. The straps 123 have an adjustable size to provide a more customized fit to the patient 102. The straps 123 may be provided with sliders 124 to allow for the size adjustment of the straps 123. The straps 123 may be removably attached to the belt 122 at the front of the patient 102. In some implementations, the straps 123 may be permanently secured to the belt 112 such that straps 123 cannot be separated from the belt without destroying the garment 110.

The smart garment 110 may include an elastic, low spring rate material that stretches appropriately to keep the device (e.g., electrodes) in place against the patient's 102 skin while the patient 102 moves. To that end, the smart garment 110 may include a conductive fiber fabric portion configured to contact the patient's skin. Preferably, the material of the smart garment 110 is lightweight and breathable. For example, the smart garment 110 may have elastic, low spring rate material composition based on a fiber content of about 10-30% (e.g., 20%) elastic fiber, 15-40% (e.g., 32%) polyester fiber, and about 0-60% (e.g., up to 48%) or more of nylon or other fiber. Additionally, the material of the smart garment may include a conductive polymer applied thereto (e.g., coated on the fibers).

In accordance with one or more examples, the smart garment 110 may be formed from an elastic, low spring rate material and constructed using tolerances that are considerably closer than those customarily used in garments. The materials for construction are chosen for functionality, comfort, and biocompatibility. The materials may be configured to wick perspiration from the skin. The smart garment 110 may be formed from one or more blends of nylon, polyester, and spandex fabric material. Different portions or components of the smart garment 110 may be formed from different material blends depending on the desired flexibility and stretchability of the smart garment 110 and/or its specific portions or components. For example, portions of the material may be formed from conductively coated fibers. As another example, the belt 122 of the smart garment 110 may be formed to be more stretchable than the back portion. According to one example, the smart garment 110 is formed from a blend of nylon and spandex materials, such as a blend of between 50-85% (e.g., 77%) nylon and 15-50% (e.g., 23%) spandex. According to another example, the smart garment 110 is formed from a blend of nylon, polyester, and spandex materials, such as 40% nylon, 32% polyester, and 14% spandex. According to another example, the smart garment 110 is formed from a blend of polyester and spandex materials, such as 86% polyester and 14% spandex or 80% polyester and 20% spandex. For example, the nylon and spandex material is configured to be aesthetically appealing, and comfortable, e.g., when in contact with the patient's skin. Stitching within the smart garment 110 may be made with industrial stitching thread.

Additionally or alternatively, example industrial sewing threads and/or yarn can form the substrate of the threads and/or yarns used in the polymer-based ECG sensing electrodes described herein. For example, industrial sewing yarns are tougher (and in some cases, larger in thickness) than other types of threads or yarns, including garment-sewing thread. In use cases, industrial yarns described herein can handle demanding conditions of industrial use, including sewing, such as multidirectional sewing, and operating at extremely high speeds. In implementations, nylon 6 and nylon 6,6 are part of the nylon family of polymers and can be used as the yarns herein. In examples, such industrial yarns can include DuPont™ Kevlar® and Dupont™ Nomex® branded threads or yarns from DuPont de Nemours, Inc., of Wilmington, Delaware, USA. In some implementations, the thread or yarn described herein include UHMWPE (ultra-high-molecular-weight polyethylene) yarns. In examples, such threads or yarns can include Spectra® branded yarn (from Honeywell International Inc. of Charlotte, North Carolina, USA) and Dyneema® branded yarn (from Avient Corporation of Avon lake, Ohio, USA). Industrial yarns described herein can be treated with a predetermined manufacturing coating that allows it to be used in a manufacturing environment. Additionally, or alternatively, the industrial yarns can be treated in order to render the yarn flame retardant and/or resistant for processes with heavy abrasion or end-uses with a high risk of ignition.

According to one example, the stitching within the smart garment 110 is formed from a cotton-wrapped polyester core thread. Additionally or alternatively, example cotton-wrapped polyester threads and/or yarn can form the substrate of basis of the threads and/or yarns used in the polymer-based ECG sensing electrodes described herein. In various embodiments, the above mentioned materials may be formed as, or coupled to, multiaxially expandable fabric portions that assist with maintaining contact of the device with the user 102. Various embodiments may include one or more multiaxially expandable fabric portions, for example, adjacent to the electrodes formed by the assembled conductive polymer coated fibers. Associated description for forming and using multiaxially expandable fabric portions are described in U.S. provisional patent application No. 63/432,465, which is incorporated herein by reference in its entirety. Maintaining proper contact between the device (e.g., ECG electrodes, therapy electrodes, and/or the connection pod) and the user 102 is particularly important in medical applications, as discussed below.

In various embodiments, the smart garment 110 may include a dock 130 configured to receive an electronic device, such as the connection pod as described in further detail herein. In some embodiments, the dock 130 is attached to the garment 110 and includes circuitry and connectors configured to couple certain garment-based devices, such as, ECG electrodes that may be permanently integrated in the garment, to the connection pod when the connection pod is attached to the dock 130. For example, integrated wiring disposed within the fabric of the garment 110 can be coupled from the ECG electrodes 112 to one or more connectors in the dock 130. These connectors can then facilitate the electrical communication of raw ECG signals from the plurality of ECG electrodes to the ECG acquisition and processing circuitry disposed within the connection pod. Some embodiments may include interconnects formed from conductive fibers instead of, or in addition to, the integrated wiring for facilitating electrical communication of raw ECG signals from the plurality of ECG electrodes to the ECG acquisition and processing circuitry disposed within the connection pod.

One of the most deadly cardiac arrhythmias is ventricular fibrillation, which occurs when normal, regular electrical impulses are replaced by irregular and rapid impulses, causing the heart muscle to stop normal contractions and to begin to quiver. Normal blood flow ceases, and organ damage or death can result in minutes if normal heart contractions are not restored. Because the victim has no perceptible warning of the impending fibrillation, death often occurs before the necessary medical assistance can arrive. Other cardiac arrhythmias can include excessively slow heart rates known as bradycardia or excessively fast heart rates known as tachycardia. Cardiac arrest can occur when a patient in which various arrhythmias of the heart, such as ventricular fibrillation, ventricular tachycardia, pulseless electrical activity (PEA), and asystole (heart stops all electrical activity) result in the heart providing insufficient levels of blood flow to the brain and other vital organs for the support of life.

Cardiac arrest and other cardiac health ailments are a major cause of death worldwide. Various resuscitation efforts aim to maintain the body's circulatory and respiratory systems during cardiac arrest in an attempt to save the life of the patient. The sooner these resuscitation efforts begin, the better the patient's chances of survival. Ventricular fibrillation or ventricular tachycardia can be treated by an external defibrillator, for example, by providing a therapeutic shock to the heart in an attempt to restore normal rhythm. To treat conditions such as bradycardia, an external pacing device can provide pacing stimuli to the patient's heart until intrinsic cardiac electrical activity returns. The smart garment 110 includes features that can monitor for and treat such conditions.

This disclosure relates to smart garments 110 that incorporate devices, such as those described above. In particular, the disclosure relates to a smart garment 110 including one or more ECG electrodes formed from a plurality of conductive fibers (e.g., conductively coated as further described by below). The ECG electrodes may be integrated into the smart garment 110 and/or removably couplable with the smart garment 110.

Advantageously, forming the ECG electrodes from individually conductively coated (or otherwise impregnated) fibers enhances comfort of wearing the smart garment and may also allow the garment to have a similar appearance and feel to normal garments. Garments having ECG electrodes formed from individually coated/impregnated conductive fibers may be easily washed without requiring removal of the ECG electrodes and/or associated electronics. Furthermore, yarn or fiber based ECG electrodes provide better flexibility than standard type ECG electrodes that are formed from rigid or semi-rigid metallic structures (e.g., silver based electrodes), and as much may better contour to the curvature of the user's anatomy, thereby reducing ECG electrode fall-off and/or noise artifacts. Furthermore, integrated or removable fiber-based ECG electrodes provide for ease of washing of the smart garment 110 without requiring timely removal of ECG electrodes from the smart garment. As such, an advantage of fabric based electrodes/sensors, formed of individually coated/impregnated fibers, is that these formed (e.g., knit, woven or otherwise having interlaced fibers) electrodes/sensors provide for improved breathability of the garment fabric in general, as well as providing for more control in placement of desired conductive fibers in selected locations of the garment. Further, the use of individually coated/impregnated fibers can provide for increased control in selected placement and/or distribution of conductive fibers in combination with non-conductive fibers (e.g., uncoated/impregnated fibers) about the body of the garment 110.

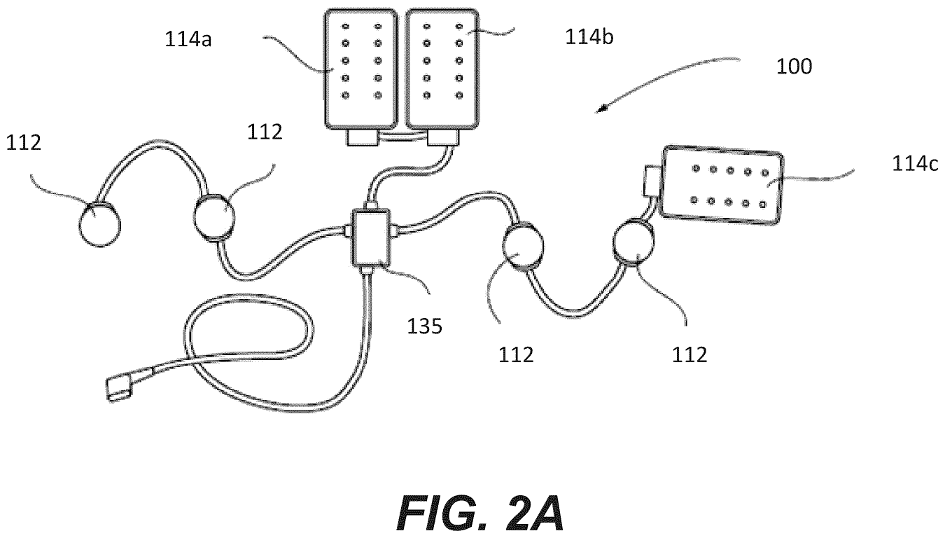

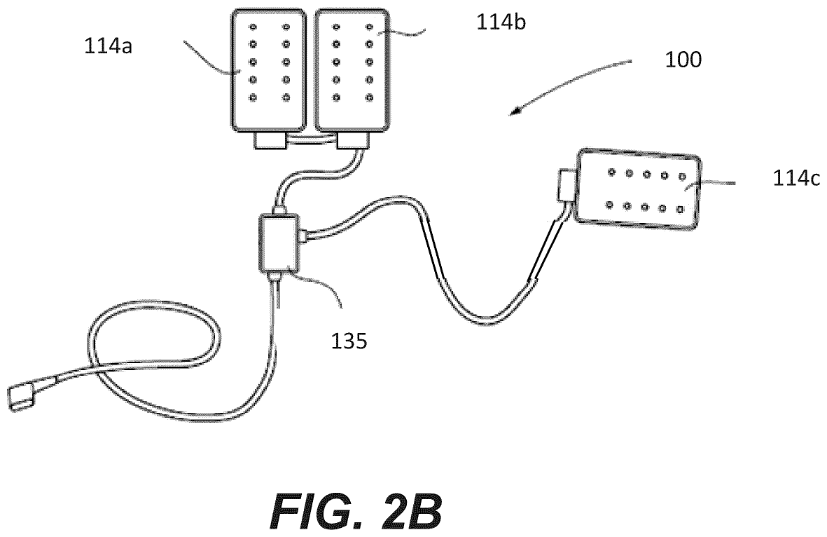

FIG. 2A schematically shows the medical device 100 (e.g., ECG electrodes 112 and/or therapy electrodes 114 as one type sensor/electrode formed from individually coated/impregnated fibers) that may be coupled to and/or integrated with the smart garment 110. As mentioned above, the smart garment 110 maintains the device 100, such as ECG electrodes 112 and/or therapy electrodes 114, in a desired contact with the user 102. For example, for ECG electrodes 112 to accurately detect ECG signals from the user 102, the ECG electrodes 112 should be in contact with the user's 102 skin. Problems frequently encountered with smart garments 110 having sensing electrodes 112 include electrode flipping (i.e., the electrode 112 contact surface becomes at least partially inverted, losing contact with the user's 102 skin) and mispositioning. Various embodiments provide ECG electrodes 112 formed from a flexible fiber integrated with the garment 110 to reduce the likelihood of electrode 112 flipping or mispositioning. Various embodiments help reduce ECG electrode 112 and/or therapy electrode 114 falloff. It is recognized that the flexibility of the electrode(s) 112 can be enhanced by using the individually coated/impregnated fibers to form/construct the electrode(s) 112.

To obtain a reliable ECG signal so that the controller and/or monitor can function effectively and reliably, it is desirable for the sensing electrodes 112 to be in the proper position and in good contact with the patient's 102 skin. The electrodes 112 can remain in a substantially fixed position and preferably do not move excessively or lift off the skin's surface. Additionally, or alternatively, the garment 110 may include a plurality of electrodes 112 on various parts of the smart garment 110. In various embodiments, a controller may determine whether the ECG electrode is in sufficient contact with the skin of a patient to obtain an ECG signal of sufficient resolution. When a particular electrode 112 is not provided a sufficient signal, a controller can determine which of the electrodes 112 are out of contact with the skin of the user and select a different one or more of the electrodes 112. As such, the ECG signal is not adversely affected with noise and is able to perform arrhythmia detection in the ECG analysis and monitoring system. Additionally, false alarms and/or shocks may be avoided.

Similarly, to effectively delivering the defibrillating energy, it is desirable that the therapy electrodes, e.g., two rear therapy electrodes 114a and 114b, and a front therapy electrode 114c (collectively therapy electrodes 114) are in a proper position, orientation, and in appropriate range of contact pressure with the patient's skin. It is desirable for the therapy electrodes 114 to be firmly positioned against the skin, minimizing electrode-skin impedance, leading to an effective and/or efficacious delivery of transcutaneous therapeutic energy to the patient's heart. Also, properly positioned therapy electrodes 114 can minimize or eliminate damage to the patient's 102 skin, such as burning, when the shock is delivered.

FIG. 2B schematically shows an implementations of the medical device 100 that may be coupled to and/or integrated with the smart garment 110 in accordance with illustrative embodiments in this disclosure. In some embodiments, the smart garment 110 may include integrated ECG electrodes 112 that are not removable from the garment 110. Accordingly, electrical cables, wires, and/or fibers may be disposed within, embedded within, weaved, knitted, sewn into, printed onto, and/or otherwise coupled with the garment 110, and may extend from various ECG electrodes 112 to the dock 130. The connection pod 135 may be configured to securably and releasable couple with the dock 130, such that the connection pod 135 is electrically coupled and communicates with the ECG electrodes 112 integrated in the garment 112. The connection pod 130 may be received directly into the receptacle of the dock 130.

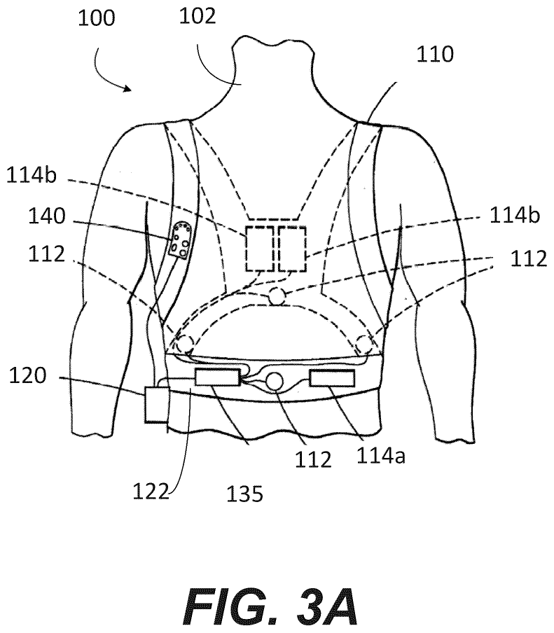

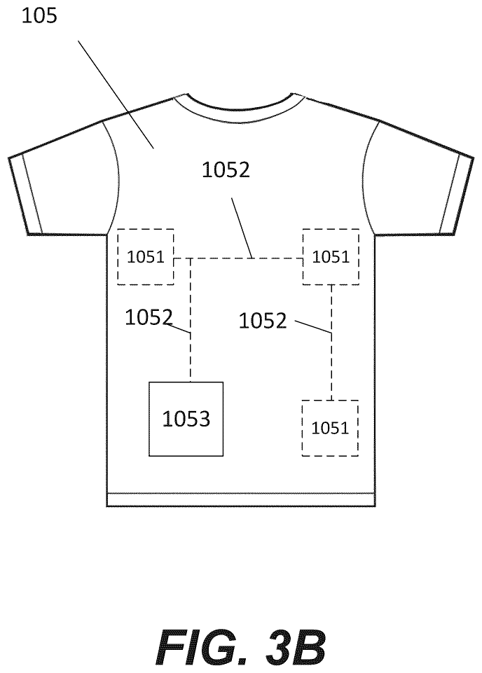

In various embodiments, the connection pod 135 communicates with the controller 120 (shown in FIG. 3A), and establishes communication between the controller 120 and the various medical devices (e.g., ECG electrodes 112 and/or therapy electrodes 114). To that end, the connection pod 135 may include an analog-to-digital converter that receives analog signals from the ECG electrodes 112 and converts them to digital signals. The ECG signals (e.g., converted to digital) are forwarded to the controller 120 for further processing. Additionally, the controller 120 may forward a signal to the connection pod 135 to activate the release of an impedance-reducing gel from the therapy electrodes 114 and/or to initiate therapy delivery via the therapy electrodes 114. Additionally or alternatively, the controller 120 may also send signals to the connection pod 135 that notify the patient 102 via tactile stimulation or sensation (e.g., vibration) on skin of the patient, before a shock is delivered by the therapy electrodes 114. To that end, the connection pod 135 may also include an electromechanical motor therein under control of the controller 120 to effectuate the vibration. As noted herein, the connection pod 135 may be a device configured to be pressed up against skin of the patient to maximize likelihood of patient discerning the tactile stimulation or sensation on patient's skin.

FIG. 3A schematically shows the patient 102 wearing the smart garment 110 in accordance with illustrative embodiments. The smart garment 110 may include one or more of the medical devices 100 (e.g., electrodes 112, 114) described with reference to FIG. 2A, FIG. 2B, or a similar system. As such, the smart garment 110 may be configured as non-invasive, wearable, ambulatory device capable of cardiac defibrillation. The smart garment 110 may be capable of and designed for moving with the patient 102 as the patient 102 goes about his or her daily routine. In one example scenario, the wearable smart garment 110 can be worn nearly continuously or substantially continuously for an extended period of time, e.g., long term use comprising, longer than 2 weeks, about a month, or about two to three months, or about three to six months, at a time. During the period of time in which the garment 110 is worn by the patient 102, the wearable defibrillator can be configured to continuously or substantially continuously monitor the vital signs of the patient 102 and, upon determination that treatment is required, can be configured to deliver one or more therapeutic electrical pulses to the patient 102. For example, such therapeutic shocks can be pacing, defibrillation, cardioversion, or transcutaneous electrical nerve stimulation (TENS) pulses.

The smart garment 110 may include various devices 100, as described earlier, including, the one or more sensing electrodes 112 (e.g., ECG electrodes), one or more of the therapy electrodes 114a and 114b (collectively referred to herein as therapy electrodes 114), a controller 120, a connection pod 135, a patient interface pod 140 (e.g., having a button), a belt 122, or any combination of these. In some examples, at least some of the devices and/or physical components of the smart garment 110 can be configured to be affixed or attached to the garment 110 (or in some examples, permanently integrated into the garment 110), which can be worn about the patient's 102 torso.

In various embodiments, the controller 120 is configured to detect a treatable arrhythmia in the patient, and in response to such detection, initiate a treatment sequence or treatment protocol. For example, such a treatment sequence or treatment protocol begins with subtle notifications to the patient 102 and steadily escalates if the patient does not respond to such notifications in a timely manner, e.g., by providing additional audible and/or tactile and/or visual notifications to the patient 102. The smart garment 110 is configured to use a combination of low volume and high volume sirens, verbal messages, and/or flashing visual notifications to get the patient's 102 attention. As the wearable defibrillator device 100 of the smart garment 110 is designed to allow patients to return to most their normal daily activities with the peace of mind that they have protection from SCA death, the smart garment 110 is configured to provide easy access to under interface functionality to allow patients 102 to respond to alerts. The smart garment 110 does not require the assistance of another person or emergency personnel for it to work. The smart garment 110 can protect patients 102 even when they are alone. In a typical situation, the entire event, from detecting a life-threatening rapid heartbeat to automatically delivering a shock, may occur in about less than one minute.

As noted, in the course of the event, a feature of various embodiments of the smart garment 110 is the series of alerts and voice prompts that keep patients 102 informed about what the device 100 is doing. These alerts let patients 102 know that the device 100 is working to protect the patient. For example, in treating a life threatening event called a ventricular fibrillation (VF) where the patient does not respond to the alarms, the treatment process may proceed in the following manner. Initially, the arrhythmia is detected, activating a vibration alert to get the patient's attention. After around 5 seconds, if the patient doesn't respond, the controller 120 initiates an audible siren alarm. For the next 20 seconds, the controller 120 sirens get louder, and the controller 120 provides audible prompts instructing the patient to “Press response buttons”. At around 30-45 seconds from the onset of the arrhythmia, if the patient still hasn't responded, the wearable defibrillator device 100 proceeds to provide a treatment shock.

In connection with the above notification sequence, in response to detecting the treatable arrhythmia, the controller 120 can send a signal to a microcontroller disposed in the connection pod 135. In response, the microcontroller in the connection pod 135 can cause a vibration motor to begin vibrating to indicate to the patient 102 that a shock is imminent. To suspend or terminate an accidental or undesirable shock, the patient 102 may engage the patient interface pod 140 or press response buttons disposed on the controller 120. In some embodiments, the patient interface pod 140 may be coupled to the smart garment 110. In some other embodiments, the patient interface pod 140 may be integrated into the controller 120, or elsewhere.

The controller 120 can be operatively coupled to the sensing electrodes 112, which can be affixed to the garment 110, e.g., assembled into the garment 110 or removably attached to the garment 110, e.g., using hook and loop fasteners. In some implementations, the sensing electrodes 112 can be permanently integrated/interlaced into the garment 110 (e.g., non-removable without destruction of the garment 110). However, in some other embodiments, the sensing electrodes 112 may be positioned with the garment 110 (e.g., by the user 102 on or otherwise within the garment 110 body). The controller 120 can be operatively coupled to the therapy electrodes 114. For example, the therapy electrodes 114 can also be assembled into the garment 110, or, in some implementations, the therapy electrodes 114 can be permanently integrated into the garment 110.

Component configurations other than those shown in FIG. 1 are possible. For example, the sensing electrodes 112 can be configured to be attached at various positions about the body of the patient 102. The sensing electrodes 112 can be operatively coupled to the controller 120 through the connection pod 135. In some implementations, the sensing electrodes 112 can be adhesively attached to the patient 102. In some implementations, the sensing electrodes 112 and at least one of the therapy electrodes 114 can be included on a single integrated patch and adhesively applied to the patient's 110 body.

The sensing electrodes 112 is a polymer-based ECG sensing electrode constructed as described herein, and configured to detect one or more cardiac signals. Examples of such signals include ECG signals, bioimpedance signals, and/or other sensed cardiac physiological signals from the patient 102. In certain implementations, the sensing electrodes 112 can include additional components such as accelerometers, acoustic signal detecting devices, and other measuring devices for recording additional parameters. For example, the sensing electrode 112 are based on poly(3,4-ethylenedioxythiophene) polystyrene sulfonate (PEDOT:PSS) material as described in detail herein. In an example scenario, a dry ECG electrode formed by fibers individually coated/impregnated with PEDOT:PSS can be placed directly on the skin and, as a result of the contact between the electrode and the skin, perspiration can accumulate on the electrode surface to provide electrical coupling with skin of the patient. In some examples, the sensing electrodes 112 can be used with an electrolytic gel dispersed between the polymer-based ECG electrode surface and the patient's skin. In implementations, advantages of dry ECG electrodes as sensing electrodes 112 include a benefit of not needing an electrolytic material dispensed between the ECG electrode surface and the patient's skin. Such dry ECG electrodes 112 can be more comfortable for continuous and/or long term monitoring applications. In various embodiments, the ECG electrodes 112 may be polarizable ECG electrodes 112.

In some examples, the therapy electrodes 114 can also be configured to include sensors configured to detect ECG signals as well as other physiological signals of the patient 102. The connection pod 135 can, in some examples, include a signal processor configured to amplify, filter, and digitize these cardiac signals prior to transmitting the cardiac signals to the controller 120. One or more of the therapy electrodes 114 can be configured to deliver one or more therapeutic defibrillating shocks to the body of the patient 102 when the smart garment 110 determines that such treatment is warranted based on the signals detected by the sensing electrodes 112 and processed by the controller 120. Example therapy electrodes 114 can include conductive metal electrodes such as stainless steel electrodes that include, in certain implementations, one or more conductive gel deployment devices configured to deliver conductive gel to the metal electrode prior to delivery of a therapeutic shock.

Some embodiments may be configured to switch between a therapeutic smart garment 110 configuration and a monitoring smart garment 110 configuration that is configured to only monitor a patient 102 (e.g., not provide or perform any therapeutic functions). For example, therapeutic components such as the therapy electrodes 114 and associated circuitry (e.g., composed of individually conductively coated/impregnated fibers) can be optionally decoupled from (or coupled to) or switched out of (or switched in to) the smart garment 110. For example, the smart garment 110 can have therapeutic elements (e.g., defibrillation and/or pacing electrodes, components, and associated circuitry) that are configured to be used when the garment 110 is placed in a therapeutic mode. In examples, the optional therapeutic elements can be physically decoupled from the smart garment 110 as a means to convert the therapeutic smart garment 110 into a monitoring for a specific use (e.g., for operating in a monitoring-only mode) or a patient 102. Alternatively, the therapeutic elements can be deactivated (e.g., by means or a physical or a software switch), essentially rendering the therapeutic smart garment 110 as a monitoring smart garment 110 for a specific physiologic purpose or a particular patient 102. As an example of a software switch, an authorized person can access a protected user interface of the smart garment 110 and select a preconfigured option or perform some other user action via the user interface to deactivate the therapeutic elements of the smart garment 110.

In accordance with one or more examples, the smart garment 110 may provide comfort and functionality under circumstances of human body dynamics, such as bending, twisting, rotation of the upper thorax, semi-reclining, and lying down. These are also positions that a patient may assume if he/she were to become unconscious due to an arrhythmic episode. The design of the garment 110 is generally such that it minimizes bulk, weight, and undesired concentrations of force or pressure while providing the necessary radial forces upon the treatment and sensing electrodes 114, 112 to ensure device functionality. A wearable defibrillator monitor may be disposed in a support holster (not shown) operatively connected to or separate from the smart garment 110. The support holster may be incorporated in a band or belt worn about the patient's waist or thigh.

FIG. 3B schematically shows an alternative of the smart garment 105 (as one further example of the smart garment 110) in accordance with illustrative embodiments in this disclosure. It should be apparent that in some embodiments, the smart garment 105 may include a shirt or other wearable garment such as but not limited to; a belt, a band, a sock, underwear (such as briefs, bra, etc.) a joint wrap (e.g., knee wrap, elbow wrap, etc.) a glove, a hat and/or pants, such as the one shown in FIG. 3B. Further, the smart garment 105 can be referred to as a smart article 105, such as but not limited to a blanket, a seat pad, a mattress pad, etc. One or more electrodes 1051 described herein may be positioned on an inside surface of the garment 110, such that the electrode 1051 surface is positioned to contact the skin of the patient. In some embodiments, further to the above, the garment 105 may include a shirt, patch, band, shirt, pants, socks, undergarment, blanket, hat, glove, and/or shoe. The various ECG electrodes 112 may be coupled using conductive interconnects 1052, as described further below. It is recognized that the electrode 1501 and/or the conductive interconnects 1502 can be formed from one or more individually coated/impregnated fibers, as further described below.

Furthermore, the conductive interconnects 1052 may couple with a module dock station 1053. As such, the module dock station 1054 an comprise a dock housing having a body with an aperture for providing access between the electrical dock connector 1055 coupled to the conductive interconnect 1052. The dock station 1053 is configured to receive an electronics module, which is electrically coupled via the electrical dock connector to the interconnect 1052.

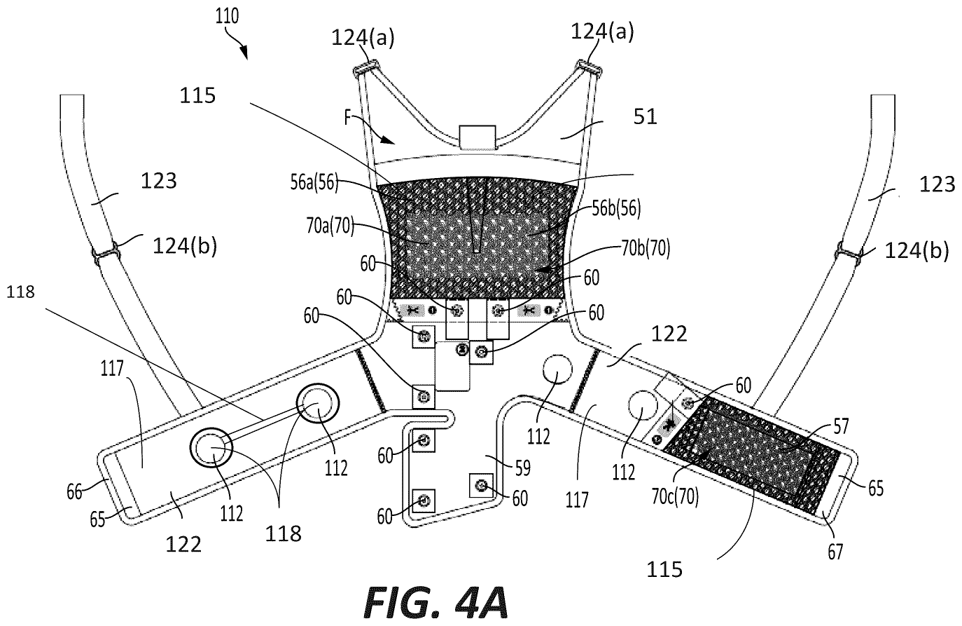

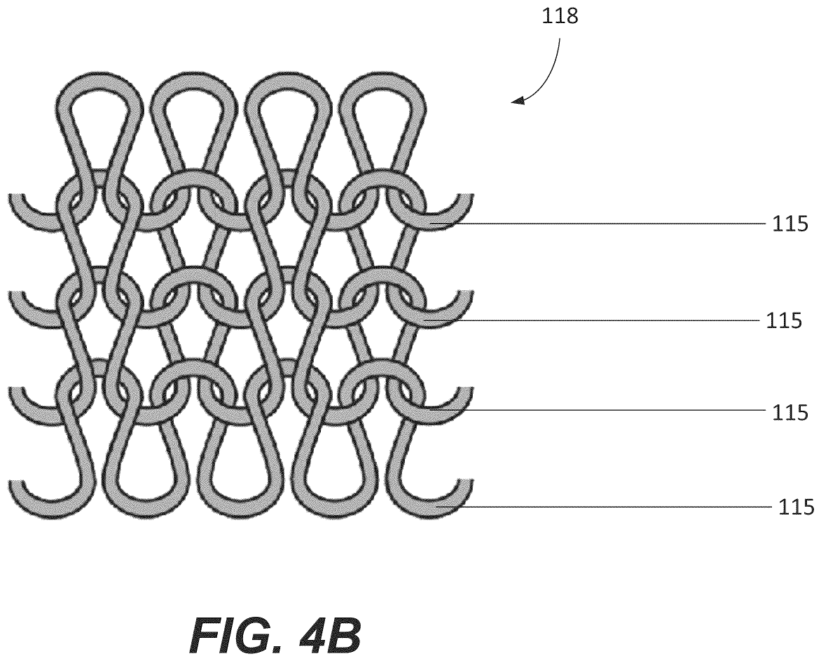

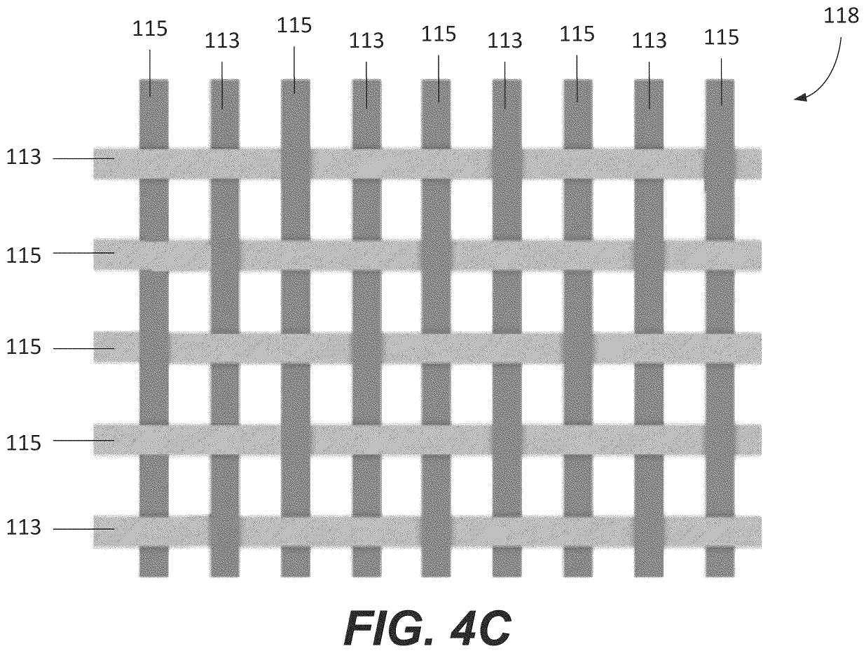

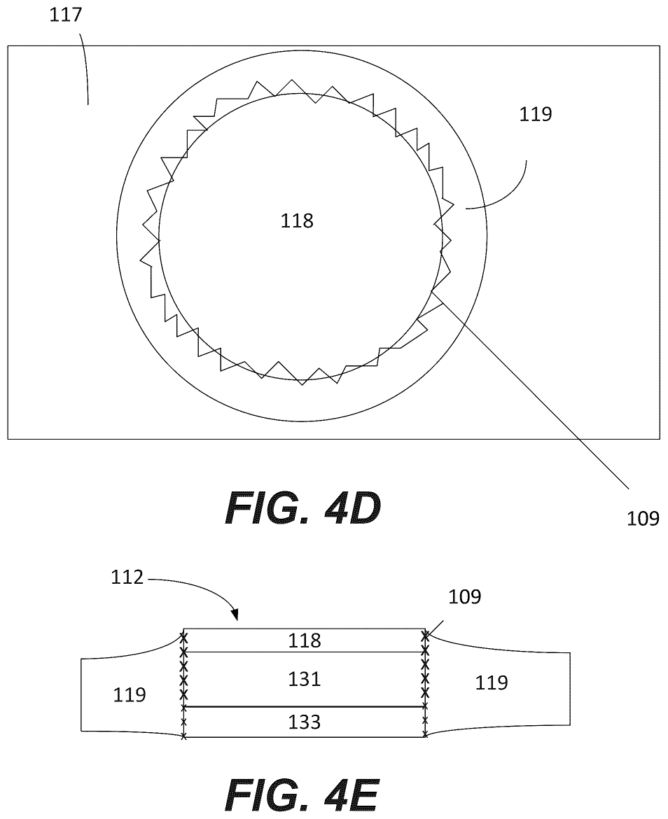

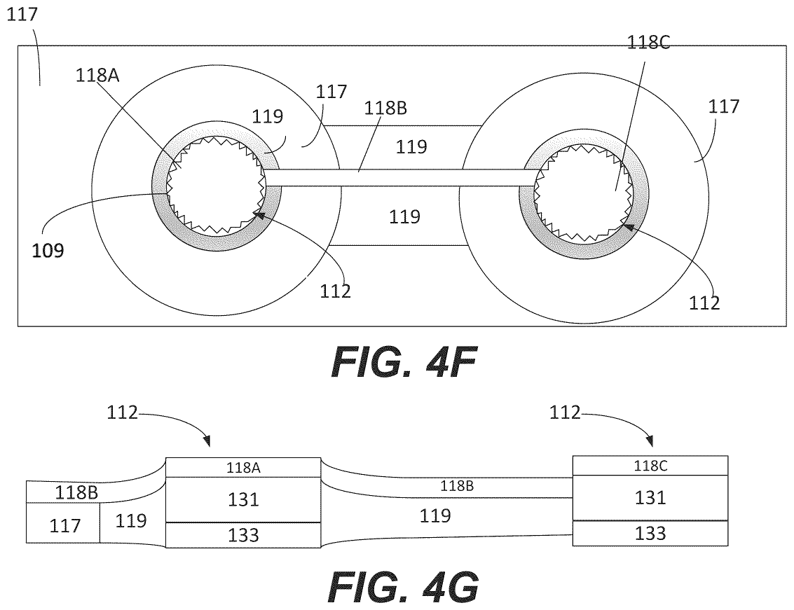

FIG. 4A illustrates an example smart garment 110 for use in non-invasive, wearable, ambulatory devices capable of cardiac defibrillation according to the present disclosure. The smart garment 110 incorporates additional improvements for enhancing the patient 102 experience of wearing the smart garment 110 for an extended period of time. The smart garment 110 examples provided herein promote comfort, aesthetic appearance, coupling between ECG electrodes 112 and the patient 102, and/or ease of use or application for older patients 102, or patients 102 with physical infirmities and/or who are physically challenged, including patients 102 with rheumatic conditions, patients with arthritis, and/or patients with autoimmune or inflammatory diseases that affect joints, tendons, ligaments, bones, and muscles of the arm and hand. Patients 102 afflicted with such conditions can properly and/or correctly don the garments 110 described herein.

Features of the smart garments 110 may also help minimize the time needed by patient 102 to assemble, don or remove the smart garment 110. Further, patients 102 benefit from such features, which can facilitate longer wear times, better patient 102 compliance, and improve the reliability of the detected physiological signals and treatment of the patient 102. These features promote ease of use, comfort and an aesthetic appearance for such patient 102 populations. For example, the features include support pockets for the therapeutic electrodes 114 that incorporate rear pocket mesh interfaces 70a and 70b, and a front pocket mesh interface 70c (collectively mesh interface 70) between the therapeutic electrodes 114 and the patient's 102 skin that is more comfortable, less abrasive, and less likely to cause irritation to the patient's 102 skin or a negative reaction.

The patient 102 may be required to wear the smart garment 110 and the components continuously or nearly continuously for extensive periods of time. Over these extensive periods of time, it is desirable to minimize any discomfort while wearing the smart garment as a result of the abrasiveness of the metal materials contained within the interfacing fabric material. As such, patients 102 benefit from a wearable cardioverter defibrillator garment 110 as described herein that includes features for enhancing the patient's 102 experience in wearing the smart garment 110 with respect to wearability and comfort of the garment with respect to the interfacing fabric materials.

These features can encourage patients 102 to wear the smart garment 110 and associated device 100 for longer and/or continuous periods of time with minimal interruptions in the periods of wear. For example, by minimizing interruptions in periods of wear and/or promoting longer wear durations, patients 102 and caregivers can be assured that the smart garment 110 is providing desirable information about as well as protection from adverse cardiac events such as ventricular tachycardia and/or ventricular fibrillation, among others. Moreover, when the patient's 102 wear time and/or compliance is improved, the device 100 can collect information on arrhythmias that are not immediately life-threatening, but may be useful to monitor for the patient's cardiac health. Such arrhythmic conditions can include onset and/or offset of bradycardia, tachycardia, atrial fibrillation, pauses, ectopic beats bigeminy, trigeminy events among others. For instance, episodes of bradycardia, tachycardia, or atrial fibrillation can last several minutes and/or hours.

The smart garments 110 herein provide features that encourage patients 102 to keep the device 100 on for longer and/or uninterrupted periods of time, thereby increasing the quality of data collected about such arrhythmias. Additionally, features as described herein, including, the mesh interfaces for the therapeutic electrode support pockets promotes better patient compliance resulting in lower false positives and noise in the physiological signals collected from ECG electrodes and other sensors disposed within the smart garment. For example, when patients 102 wear the device for longer and/or uninterrupted periods of time, the device 100 tracks cardiac events and distinguishes such events from noise over time.

The improvements incorporated in the smart garment 110 may provide comfort and wearability to the patient by utilizing a mesh interface 70 made from a layer or layers of fabric material incorporating a reduced amount of conductive metal content. Various embodiments may form the entirety, or portions, of the mesh interface 70 from a conductive fiber. Accordingly, various embodiments may provide reliable contact between the therapeutic electrodes 114 and the patient 102 for treatment. The fabric material of the mesh interface 70 may additionally, or alternatively, incorporate component materials that have a soft, comfortable feel on the patient's 102 skin and are configured to wick moisture away from the patient's 102 skin. The fabric material of the mesh interface 70 may be less abrasive to the patient's 102 skin and less likely to cause irritation to the patient's skin or a negative reaction.

In accordance with one or more examples, the smart garment 110 is provided to keep the electrodes 114, 112 of an electrode assembly 100 associated with a wearable cardiac therapeutic device in place against the patient's 102 body while remaining comfortable to wear. In particular, the electrode assembly 100 may include the plurality of ECG sensing electrodes 112 configured to sense ECG signals regarding a cardiac function of the patient 102 and the plurality of therapy electrodes 114 configured to deliver transcutaneous defibrillation shocks or transcutaneous pacing pulses or other types of therapeutic electrical pulses, to the patient's 102 heart. For example, the pacing pulses comprises current of one or more of the pacing pulses between 0.1 mA and 300 mA. It is to be appreciated that the smart garment 110 described herein may be utilized in connection with a wearable of any suitable type or configuration. As shown in FIG. 4A, the smart garment 110 may be provided in the form of a vest or harness having a back portion 51 and sides extending around the front of the patient 102 to form the belt 122. In various embodiments, the back portion 51, the belt 122, or a portion thereof may be formed from one or more conductive fibers.

FIG. 4B schematically shows a knitted conductive fabric portion 118 of the smart garment 105, 110 in accordance with illustrative embodiments in this disclosure. The conductive fabric portion includes one or more conductive polymer coated fibers 115, recognizing that the conductive polymer coated fibers 115 can also be referred to as polymer impregnated fibers 115. As such, it is recognized that the applied conductive polymer (e.g., as described herein) can be used to coat or otherwise impregnate the individual fibers 115, to render the individual fiber 115 conductive or otherwise improve the conductivity of the individual fiber 115. For ease of discussion, various embodiments may refer to the conductive polymer coated fibers as polymer coated fibers 115 or coated fibers 115. It is also recognized that the applied conductive polymer can be applied to the exterior surface (e.g., as a coating) of each individual fiber 115 and/or can be applied to an interior (e.g., below the exterior surface as an impregnation) of each individual fiber 115. As such, as further provided herein, the terms “coating”, “coated”, “impregnated” and “impregnating” can be used interchangeably. In some embodiments, each of the one or more conductive polymer coated fibers 115 may be a single fiber or a multifiber. For example, each of the fibers 115 may be a multifiber, such as a yarn, which has a plurality of single fibers that are intertwined. In various embodiments, the single fiber 115 or multifiber 115 may be coated prior to assembly into the fabric portion 118. In some embodiments, single fibers 115 may be coated prior to intertwining into a multifiber 115. Alternatively, some embodiments may coat the multifiber 115 after intertwining. In further embodiments, the single fibers 115 may be coated before intertwining to become a multifiber 115, and then coated again after intertwining.

In various embodiments, all, or portions of, the smart garment 105, 110 may be assembled by knitting (e.g., one example embodiment of interlacing). In particular, a plurality of conductive fibers 115 (e.g., conductively coated) may be assembled by knitting. In the example of FIG. 4B, all of the fibers are conductive fibers 115. However, it should be understood that various embodiments of the conductive fabric portion 118 may interlace conductive fibers 115 (e.g., individually impregnated with conductive polymer) with non-conductive fibers 113. The plurality of conductive fibers 115 may define a conductive fabric portion 118 that is coupled with a non-conductive fabric portion.

FIG. 4C schematically shows a weaved conductive fabric portion 118 of the smart garment 110 in accordance with illustrative embodiments in this disclosure. In particular, a plurality of conductive fibers 115 may be assembled by weaving (e.g., one example embodiment of interlacing). As shown, the conductive fibers 115 (e.g., individually impregnated with conductive polymer) may be interlaced with the non-conductive fibers 113. Among other ways, the conductive fibers 115 and the non-conductive fibers 113 may alternate every other one, e.g., as shown with the vertical fibers. Additionally, or alternatively, a plurality of conductive fibers 115 may be grouped together between non-conductive fibers 113, e.g., as shown with the horizontal fibers.

The configurations shown in FIGS. 4B-4C are not intended to limit various embodiments of the invention. Instead, FIGS. 4B-4C are merely exemplary and intended to show a few of many examples of the conductive fabric portion 118 (e.g., including one or more fibers 115 individually impregnated with conductive polymer). One skilled in the art should understand that there are a variety of ways to interlace conductive fibers 115 and non-conductive fibers 113 within the scope if this disclosure. Advantageously, by interlacing conductive fibers 115 and non-conductive fibers 113, the comfort of the smart garment 105, 110 may be enhanced. It should be also understood that although all of the fibers of FIG. 4B are conductive, this is merely for exemplary purposes, and is not intended to limit knitting or weaving of smart garments 110 to use exclusively with conductive fibers 115. Smart garments may be knitted, weaved, embroidered, or otherwise assembled/interlaced using a variety of conductive fibers 115 and/or non-conductive fibers 113.

FIG. 4D schematically shows a magnified view of a portion of the smart garment 105, 110 of FIG. 4A. In particular, FIG. 4D shows a top view of the ECG electrode 112 formed from the conductive fabric portion 118 (e.g., including one or more fibers 115 individually impregnated with conductive polymer). The conductive fabric portion 118 may be weaved or knitted, as discussed previously. In addition to the conductive fabric portion 118, the smart garment 110 may be comprised of an elastic, low spring rate fabric material that stretches appropriately to keep the electrodes 112 in place against the patient's 102 skin and is lightweight and breathable. The conductive fabric portion 118 may be stitched to the first fabric portion 117 and/or some other intermediary stretchable fabric portion 119. In some embodiments, the electrodes 112 can be removably attached, e.g., via hook and loop fasteners or snap connectors, to attachment regions of the garment 105, 110.

The component materials of the base fabric material of the conductive fiber 115 may be chosen for functionality, comfort, and biocompatibility. The component materials may be configured to wick perspiration from the skin. For example, the fabric material may comprise a tricot fabric, the tricot fabric comprising nylon and spandex materials. The tricot fabric may comprise approximately 65%-90% nylon material, or more particularly 70%-85% nylon material, or more particularly 77% nylon material. These base materials may be coated with a conductive polymer to form a conductive fabric portion 118 (e.g., including one or more fibers 115 of the base material individually impregnated with conductive polymer). In some embodiments, a first fabric portion 117 that is non-conductive may be formed from the previously referenced fabrics. In some embodiments, the first fabric portion 117, or other non-conductive portion, may form a majority of the garment 105, 110.

FIG. 4E schematically shows a cross-section of FIG. 4D. In some implementations of the garment 105, 110 described above, the first fabric portion 117 and/or the stretchable fabric portion 119 may be coupled with the conductive fiber fabric portion 118 at an interface 121 (e.g., a seam 109, such as a line along which the two fabric portions are sewn/stitched together, for example using conductive fibers 115 and/or nonconductive fibers 113). In some embodiments, the stretchable fabric portion 119 at least partially surrounds the polymer-based ECG sensing electrodes 112. A yield strain ratio of the stretchable fabric portion relative to the polymer-based ECG sensing electrodes ranges between about 1.1 to about 6.0. Thus, the stretchable fabric portion that surrounds the electrodes 112 may be about 1.1 to about 6 times as flexible as the ECG sensing electrodes 112.

In examples, the first fabric portion 117 and the conductive fiber fabric portion 118 are both knitted portions. For example, such first fabric portion 117 can be manually joined to the conductive fiber fabric portion 118 using a flatbed knitting machine. For example, such first fabric portion 117 can be machine joined to the conductive fiber fabric portion 118 using a linking machine, e.g., a high-speed linking machine. In examples, linking includes seaming and/or attaching pieces of the foregoing fabric portions together after the pieces have been knitted on a flat-bed knitting machine. In examples, a slacker course of loops of yarn can be created on the linking machine, which connects the two pieces of fabric together.

For example, such first fabric portion 117 can be manually joined to the conductive fiber fabric portion 118 using one or more cut and sew methods. For examples, such methods include seaming, such as an open seam (e.g., where the seam allowance, the piece of fabric between the edge of the material and the stitches, is visible) or a closed seam method (e.g., incorporates the seam allowance within the seam finish, making the seam allowance invisible). Seams 109 can include plain seams, double-stitched seams, French seams, bound seams, Flat-felled seams, Welt seams, or lapped seams. In examples, a bias tape (e.g., a narrow strip of fabric) can be folded over an exposed seam to secure and hide edges. In examples, a zigzag stitch can be implemented along a raw edge of the seam to secure the edges and prevent fraying. In examples, a faux overlock stitch can be implemented in the seam. In examples, a reinforced straight stitch can be implemented in the seam. In examples, hemming can be implemented in the seam. In examples, depending on the yarn material and/or make up, ultrasonic bonding techniques, including creation and channeling of high frequency vibratory waves that cause a rapid buildup of heat can be used to implement joins. An example of such a device includes the SeamMaster® General Purpose Ultrasonic Sewing Machine from Sonobond Ultrasonics of West Chester, PA, USA. For examples, fabric materials suited for ultrasonic joins includes thermoplastic fabric and/or film materials including acrylic, nylon, polyester, polyethylene, polypropylene, polyvinylchloride and urethane. Accordingly, one or both of first fabric portion 117 and conductive fiber fabric portion 118 comprises materials including thermoplastic fabric and/or film materials including acrylic, nylon, polyester, polyethylene, polypropylene, polyvinylchloride and urethane. In some embodiments, the first fabric portion 117 may be interwoven, adhered, glued, sewn, chemically welded, and/or heat welded with the conductive fiber fabric portion 118 at the interface 121.

In various embodiments, the first fabric portion 117 and conductive fiber fabric portion 118 are integrated into the knitting, e.g., incorporated into a knitting machine software program such that the knitted resulting fabric transitions from multiaxially expandable fabric to non-multiaxially expandable fabric zones in the same fabric material. In this regard, first fabric portion 117 and conductive fiber fabric portion 118 are implemented on a common fabric. For example, a computer-based knitted machine from, e.g., Stoll of the Karl Mayer Group based and based in Dayton, OH, USA, can be used to knit such materials as described herein. In this regard, as a first step, a knitting program comprising computer-readable code executable by a computer coupled to the knitting machine can be implemented to cause the fabric to knit a first non-multiaxially expandable fabric zone in a first, predetermined region of the fabric. As a second step, the computer-readable code can instruct the knitting machine to cause multiaxially expandable fabric zones in a second predetermined region of the fabric. In regions of the common fabric where it is desirable to implement the conductive fiber fabric portion 118, techniques for developing the conductive fiber fabric portion 118 as described herein can be implemented through the knitting program. In such methods, regions of first fabric portion 117 and regions of conductive fiber fabric portion 118 are disposed on a same fabric, and as such the different regions result from use of different knitting techniques to create the desired regions.

The conductive fiber fabric portion 118 may be made of the same or similar materials as the first fabric portion 117. However, as discussed below, the conductive fiber fabric portion 118 includes one or more fibers having a conductive polymer applied thereto (e.g., coated/impregnated thereon). In some other embodiments, the first fabric portion 117 and the multiaxially expandable portion 118 may be formed of different materials. As shown in FIG. 4A, the smart garment 105, 110 may be provided in the form of a vest or harness having a back portion 51 and sides extending around the front of the patient 102 to form the belt 122. In various embodiments, the back portion 51, the belt 122, or a portion thereof may be formed as a multiaxially expandable fabric. In some embodiments, the conductive fiber fabric portion 118 may be formed as electrodes 112 to assist with reducing the likelihood of electrode flipping, rotation, twisting, or other undesirable movement, as patients 102 with torsos of various shapes and/or sizes perform their normal activity throughout the course of the day.

To that end, in various embodiments an ECG electrode 112 may be formed by an exposed or lop layer of the conductive fiber fabric portion 118. In some embodiments, the conductive fiber fabric portion 118 is overlayed over a shock absorbing portion 131 and/or a support panel 133. The foam layer assists with achieving a more uniform pressure on the skin, thereby improving the contact between the electrode and the body. This contact assists with reducing noise and artifacts in the received ECG signal. The expandable fabric portion 118 is then coupled with the remainder of the garment 110 by directly coupling with the first fabric portion 117 and/or an intermediary stretch fabric portion 119. In various embodiments, the stretch fabric portion 119 may be a multiaxially expandable portion.

The smart garment is configured to maintain the electrical contact between the one or more of the plurality of ECG electrodes 112 and skin of the patient at least by pressing the one or more of the plurality of ECG electrodes against the skin of the patient of the smart garment at a predetermined range of pressures. In embodiments, the smart garment 110 may be configured to maintain a contact pressure between the patient's 102 skin and the ECG electrode 112 of between about 5 and about 150 mm Hg (e.g., 75 mm Hg). In embodiments, the smart garment 110 may be configured to maintain a contact pressure between the patient's 102 skin and an ECG electrode 112 of between about 5 and about 150 mm Hg (e.g., 50 mm Hg). In embodiments, the garment 110 may be configured to maintain a contact pressure between the patient's 102 skin and an ECG electrode 112 of between about 5 and about 50 mm Hg (e.g., 25 mm Hg). In embodiments, the garment 110 is configured to maintain a contact pressure between the patient's 102 skin and an ECG electrode 112 of between about 5 and about 40 mm Hg (e.g., 15 mm Hg). In embodiments, garment 110 may be configured to cause to maintain a contact pressure between the patient's 102 skin and an ECG electrode 112 of between about 0.1 psi and about 0.8 psi (e.g., 0.6 psi). In embodiments, the garment 110 may be configured to maintain a contact pressure between the patient's 102 skin and an ECG electrode 112 of between about 0.1 psi and about 2 psi (e.g., 1 psi). In embodiments, the garment 110 may be configured to maintain a contact pressure between the patient's 102 skin and an ECG electrode 112 of between about 0.1 psi and about 3 psi (e.g., 1.5 psi).

The smart garment 110 is configured to maintain the electrical contact between the one or more of the plurality of therapy electrodes 114 (114a, 114b, and 114c) and skin of the patient at least by pressing the one or more of the plurality of ECG electrodes 112 against the skin of the patient 102 of the smart garment 110 at a predetermined range of pressures. In embodiments, the garment 110 may be configured to maintain a contact pressure between the patient's 102 skin and a therapy electrode 114 of between about 5 and about 150 mm Hg (e.g., 75 mm Hg). In embodiments, the smart garment 110 may be configured to maintain a contact pressure between the patient's 102 skin and a therapy electrode 114 of between about 5 and about 150 mm Hg (e.g., 50 mm Hg). In embodiments, the smart garment 110 may be configured to maintain a contact pressure between the patient's 102 skin and a therapy electrode 114 of between about 5 and about 50 mm Hg (e.g., 25 mm Hg). In embodiments, the smart garment 110 may be configured to maintain a contact pressure between the patient's 102 skin and a therapy electrode 114 of between about 5 and about 40 mm Hg (e.g., 15 mm Hg). In embodiments, the smart garment 110 may be configured to maintain a contact pressure between the patient's 102 skin and a therapy electrode 114 of between about 0.1 psi and about 0.8 psi (e.g., 0.6 psi). In embodiments, the smart garment 110 may be configured to maintain a contact pressure between the patient's 102 skin and a therapy electrode 114 of between about 0.1 psi and about 2 psi (e.g., 1 psi). In embodiments, the smart garment 110 may be configured to maintain a contact pressure between the patient's 102 skin and a therapy electrode 114 of between about 0.1 psi and about 3 psi (e.g., 1.5 psi).

FIG. 4F schematically shows a magnified view of another portion of the smart garment 105, 110 of FIG. 4A. FIG. 4G schematically shows a cross-sectional view of FIG. 4F. As shown, the smart garment 105, 110 may include multiple electrically coupled conductive fiber fabric portion sections 118A, 118B, and/or 118C. Conductive fiber fabric portion section 118B serves as an interconnect 118B that keeps the ECG electrodes 112 in communication with another. The polymer fiber interconnect 118B is formed by assembling the plurality of the individually polymer coated conductive fibers 115 in a longitudinal pattern between two of the plurality of polymer-based ECG sensing electrodes 112. Although the interconnect 118B is shown as a top exposed layer, not all embodiments are limited thereto. Indeed, in some embodiments, the interconnect 118B may be a sub-surface layer underneath the first fabric portion 117 and/or stretchable fabric portion 119. Interconnects 118B may electrically couple a plurality (or all) of the ECG electrodes 112 of the smart garment 110. Furthermore, the interconnects 118B may electrically couple the electrodes 112 with the dock 130 and/or the controller 120. Thus, the interconnects 118B may facilitate electrical communication of raw ECG signals from the plurality of ECG electrodes to the ECG acquisition and processing circuitry. In some embodiments, the interconnects 118B may include a single conductive fiber 115.

Returning to FIG. 4A, the ends 66, 67 of the belt 122 may be connected at the front of the patient 102 by a closure mechanism 65. The smart garment 110 may further include two straps 123 connecting the back portion 51 to the belt 122 at the front of the patient 102. In examples, one or more devices 100 as described herein can be disposed on one or both straps 123. For example, the devices 100 may be permanently coupled to one or both straps 123. For example, the devices may be removably coupled to one or both straps 123. In examples, one or more ECG electrodes 112 as described herein can be disposed on one or both straps 123. For example, the ECG electrodes 112 may be permanently coupled to one or both straps 123. For example, the ECG electrodes 112 may be removably coupled to one or both straps 123. The straps 123 have an adjustable size to provide a more customized fit to the patient 102. In some embodiments, the straps 123 may be include a conductive fiber fabric portion 118. Thus, push devices 100 disposed in the smart garment (e.g., on one or both straps 123 or elsewhere on smart garment 110) may also be electrically coupled via a conductive fiber 115. First strap sliders 124a may be provided to connect the straps 123 to the back portion 51 of the smart garment. Second strap sliders 124b may be provided along the straps 123 to facilitate size adjustment of the straps 123. The straps 123 with such conductive fiber fabric portion 118 may be removably attached to the belt 122 at the front of the patient 102. In implementations, the straps 123 with such conductive fiber fabric portion 118 may be permanently secured to the belt 122 at the front of the patient 102, such that the strap 123 cannot be separated from the belt 122 without destruction of the smart garment 110.

The smart garment 105, 110 may be configured for one-sided assembly of the electrode assembly 100 onto the smart garment 105, 110 such that the smart garment 105, 110 does not need to be flipped or turned over to properly position the therapy electrodes 114 and the sensing electrodes 112 on the smart garment 105, 110. The inside surface of the back portion 51 of the smart garment 105, 110 includes one or more pocket(s) 56B, 56C (together 56) for receiving one or two therapy electrodes 114 to hold the electrode(s) 114 in position against the patient's 102 back. The one or more pockets 56 includes a mesh interface 70 (or mesh interfaces 70a, 70b) incorporating a plurality of conductive fibers for transmitting electrical energy from the therapy electrode towards the patient's skin. In examples, the one or more pockets 56 can be configured to include dielectric fibers comprising at least one nonmetallic material and a plurality of conductive fibers or particles therein, as well as a plurality of openings defined therein. The garment region forming the rear of the pockets 56 (e.g., the fabric opposing the mesh portion 70) may include a multiaxially expandable portion 119 configured to push the therapy electrode towards the patient's skin when the garment region is stretched along traverse orientations (e.g., orientations substantially perpendicular to the axis oriented towards the patient).

Alternatively, or additionally, in some embodiments, one or more pockets as described above for receiving the therapy electrodes 114 may be accessible from an outside surface of the smart garment 110 (e.g., the surface of the garment that is facing away from skin of the patient) rather than an inside surface of the garment. In such implementation, the one or more pockets includes a mesh interface (similar to mesh interface 70 or mesh interfaces 70a, 70b as described above) incorporating a plurality of dielectric fibers comprising at least one nonmetallic material and a plurality of conductive fibers or particles therein, as well as a plurality of openings defined therein. The garment region forming the rear of the pockets 56 may include a multiaxially expandable portion 118 configured to push the therapy electrode towards the patient's skin when the garment region is stretched along traverse orientations.

The mesh interface 70 is configured to physically separate the metallic therapy electrode(s) 114 from the skin of the patient 102 while allowing a conductive gel that may be automatically extruded from a plurality of holes 61 in the electrode(s) 114 to easily pass through to the skin of the patient 102. The forces applied to the electrode(s) 114 by the mesh interface 70, in addition to the use of the conductive gel, may help ensure that proper contact and electrical conductivity with the patient's 102 body are maintained, even during body motions. The mesh interface 70 also maintains electrical contact between the electrode(s) 114 through the material of the mesh interface 70 before the conductive gel is dispensed, which allows for monitoring of the therapy electrode(s) 114 to ensure that the electrode(s) 114 are positioned against the skin such that a warning may be provided by the smart garment 110 if the therapy electrode(s) 114 is not properly positioned. Another pocket, front pocket 57 including a mesh interface 70c according to the same construction is included on an inside surface of the belt 122 for receiving a front therapy electrode 114c and holding the electrode 114 in position against the patient's 102 left side. Similar to the configuration of the rear pockets, the garment region forming the rear of the front pocket 57 may include a multiaxially expandable portion 118 configured to push the front therapy electrode towards the patient's skin when the garment region is stretched along a circumferential orientation (e.g., in a direction along the circumference of torso of the patient in an anatomical axial plane). In various embodiments, the mesh interface 70 of any of the pockets 56, 57 may be formed as a conductive fiber fabric portion 118. Accordingly, the openings of the mesh may be configured to expand as the belt 122, back portion 51, and/or pockets 56, 57 are stretched by the patient's 102 body.

After assembly of the therapy electrode(s) 114 into the respective pocket(s) 56, 57, the pocket(s) 56, 57 are closed on the smart garment 110, by a fastener or fasteners 60, such as a button or snap. Two rear pockets 56B and 56C, and one front pocket 57 are shown corresponding to the two rear therapy electrodes 114a and 114b, and front therapy electrode 114c. In other implementations, fewer or more rear or front pockets and/or therapy electrodes may be provided. For example, the garment 110 can include two rear pockets and two front pockets, these pockets configured to receive two rear therapy electrodes and two front therapy electrodes. For example, the garment 110 can include three rear pockets and three front pockets, these pockets configured to receive three rear therapy electrodes and three front therapy electrodes. In such examples, the rear or front pockets can include corresponding mesh interface as described herein. Some or all of the pockets may include a multiaxially expandable portion.

The back portion 51 and the belt 122 of the smart garment 110 may further incorporate attachment points 58 for supporting the sensing electrodes 112 in positions against the patient's 102 skin in spaced locations around the circumference of the patient's 102 chest. The attachment points 58 may include hook-and-loop fasteners for attaching ECG sensing electrodes 112 having a corresponding fastener disposed thereon to the inside surface of the belt 122. The attachment points 58 may be color coded to provide guidance for appropriately connecting the sensing electrodes 112 to the smart garment 110. Additionally, or alternatively, one or more of the ECG sensing electrodes can be permanently integrated into the belt 122 of the smart garment 110, e.g., such that they cannot be removed/replaced by a patient during use. The smart garment 110 may further be provided with a flap 59 extending from the back portion 51.

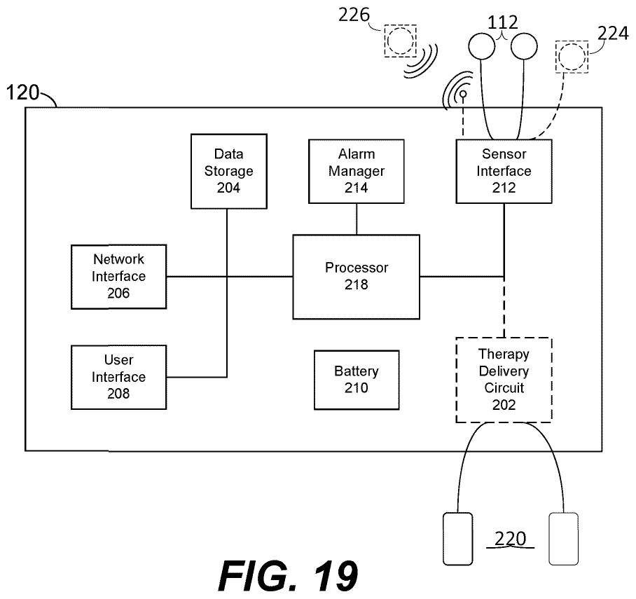

The flap 59 and the back portion 51 include fasteners 60 for connecting the flap 59 to the inside surface of the back portion 51 to define a pouch or pocket for holding a connection pod 135. The connection pod 135 may include processing and/or vibrational circuitry of the electrode assembly 100. For example, the connection pod 135 can include ECG acquisition and conditioning circuitry configured to receive ECG signals from the plurality of ECG sensing electrodes 112, amplify the signals, condition (e.g., using filter circuits) to remove noise, and sample the signal to produce a digitized ECG signal corresponding to the analog ECG input. In examples, the connection pod 135 can also include vibrational circuitry configured to receive an input from a controller (e.g., controller 120 shown in FIG. 19 below) and provide the patient 102 a vibrational alarm or notification as appropriate. The outer surface of the belt 122 may incorporate a schematic imprinted on the fabric for assisting the patient or medical professional in assembling the electrode assembly 100 onto the smart garment 110. Portions of the garment 110 of the flap 59, and/or portions adjacent to the fasteners 60 and the attachment points 58 may also be formed as a multiaxially expandable fabric. In examples, the garment fabric in the region around the location of the connection pod 135 can include multiaxially expandable fabric configured such that the connection pod 135 is caused to push against the body of the patient when the multiaxially expandable fabric in the region is subject to transverse forces (e.g., stretch forces in directions that are substantially perpendicular to an axis oriented towards the patient's skin).

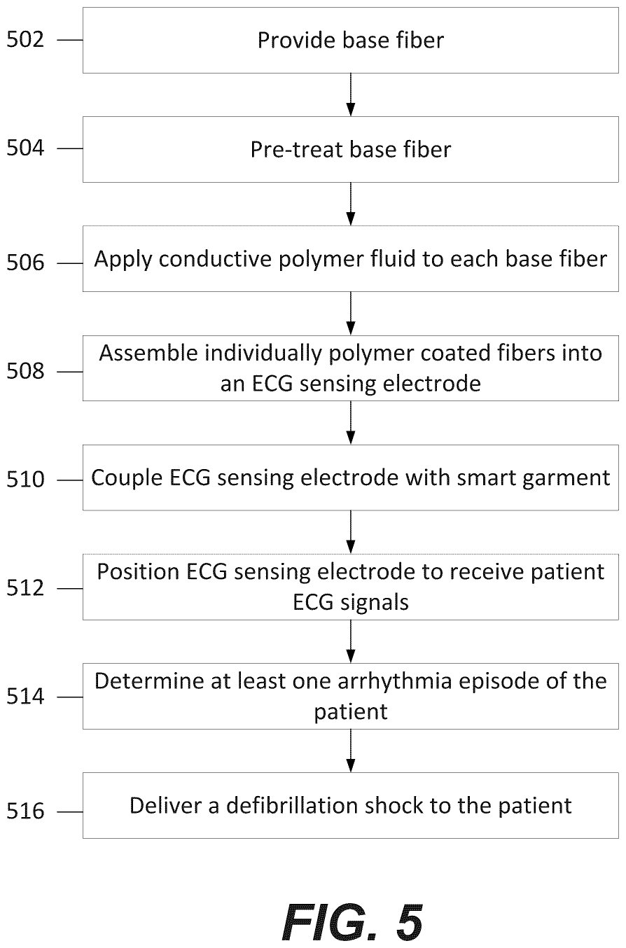

FIG. 5 shows an example process 500 of making and using the smart garment 105, 110 in accordance with illustrative embodiments. The process begins at step 502, which provides a base fiber 111. The base fiber 111 may be a natural or synthetic fiber. In some embodiments, the base fiber 111 may be non-conductive. Among other things, the base fiber 111 may be formed from cationic polyester (PES), polyamide 6 (PA 6), polyamide 66 (PA 66), nylon 6, and/or nylon 6,6. The base fiber 111 may also be formed from synthetic fibers such as polyester or even natural fibers such as cotton, silk, and/or wool. In various embodiments, the base fiber 111 may also be a conductive fiber. Accordingly, illustrative embodiments may improve the conductivity of a base conductive fiber 111. For instance, carbon-contained fibers are already conductive, but their resistance is quite high. To lower their resistance and/or make them biocompatible, the base conductive fiber 111 can be coated using biocompatible conductive inks. The base fiber 111 may also include yarn. As known by those of skill in the art, yarn is a multifiber. In various embodiments the base fiber 111 may be a single fiber or a multifiber.

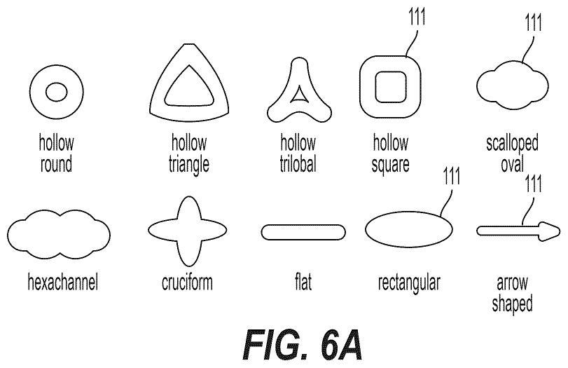

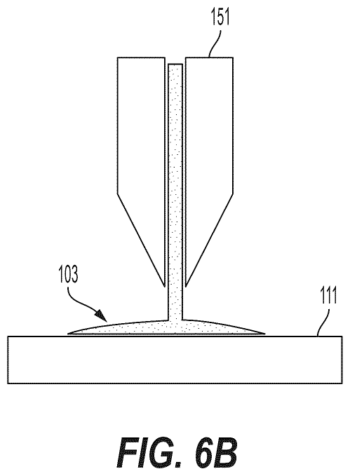

FIG. 6A schematically shows a variety of cross-sectional fiber shapes in accordance with illustrative embodiments. The cross-sectional geometry of the base fiber 111, also referred to as the substrate, can influence coating/impregnation adhesion properties of the conductive polymer fluid to the fiber 111 (e.g., surface and/or interior of a single strand or multistrand fiber 111). In examples, regular or irregular cross-sectional yarns are used. In examples, the cross-section of the yarn is configured to maximize adhesion/impregnation of the conductive polymer fluid to the fiber 111. Therefore, illustrative embodiments select an appropriate cross-sectional geometry of the base fiber 111. Shaped fibers refer to fibers 111 that have a cross-section other than round. Such fibers 111 can be found in both natural fibers and synthetic fibers. Synthetic shaped fibers 111 may be used to achieve functional properties such as softness, luster, abrasion resistance, coefficient of friction, thermal comfort, bending rigidity, and liquid or moisture transfer. The cross-sectional shape of the fibers 111 that are made through melt/wet/dry spinning can be varied by changing the spinneret hole shape. Various embodiments may begin with fibers having a predetermined cross-sectional shape, such as circular, rectangular, trilobal, hollow round, hollow triangle, hollow trilobal, hollow square, scalloped oval, hexachannel, cruciform, flat, and/or arrow shaped.

The process then proceeds to step 504, which can optionally pretreat the base fiber 111 to assist with adhesion of the conductive polymer fluid applied (e.g., coating, impregnation, etc.) in subsequent steps. FIG. 6B schematically shows plasma 103 treating the base fiber 111 in accordance with illustrative embodiments. In some embodiments, the surface of the fiber 111 is plasma 103 treated. In various embodiments, the plasma 103 treatment may use a source of compressed gas 151, which may be a process gas and/or a cooling gas. More particularly, some embodiments use open-air atmospheric pressure plasma 103 pretreatment. Plasma treatment modifies the surface properties of the fiber, to enhance the adhesion of the conductive polymer fluid to the base fiber 111 when applied. With plasma 103 cleaning, fiber 111 surfaces are cleaned and sterilized, while plasma 103 activation makes later adhesion of glues and coatings possible. Plasma 103 cleaning may help increase surface energy, making the fiber 111 easier to bond to. Static charges also build up on the fiber 111 that may attract dust and other contaminants that may interfere with effective bonding. High velocity ionized plasma 103 particles break up and remove surface contaminants, exposing a stable bonding surface of the base fiber 111.

Some other embodiments may use atmospheric plasma 103 surface etching. Plasma surface etching is a type of plasma 103 treatment used to increase the surface area of the fiber 111 on the microscopic scale. The surface of the fiber 111 is etched with a reactive process gas. Material from the surface is etched away, converted to the gas phase and removed by the vacuum system. The surface area is greatly increased, raising the surface energy and making the material easily wettable. Plasma surface etching is used before printing, gluing and painting and is particularly useful for processing of e.g., POM and PTFEs. Atmospheric plasma treatment is ideal for in-line processing and is very effective at activating and cleaning the surface of the fiber, increasing the adhesion characteristics and making it easier to coat. The atmospheric plasma nozzle can be clamped to a fixed point or to a simple multi-axis robot making it ideal for industrial applications.

For example, in various embodiments, the fiber 111 may be plasma 103 treated to roughen the surface. In various embodiments, the process proceeds to step 506 within about 1 hour to about 72 hours, or between about 6 hours and about 48 hours, or between about 12 hours and about 24 hours, or about 24 hours, prior to applying a conductive polymer fluid. Preferably, the process proceeds to step 506 within 4 hours of step 504.

The inventors determined that plasma 103 treating the yarns 111 prior to coating them with PEDOT (e.g., an example of the conductive polymer fluid) was found to have little to no significant effect on the quality or performance of the electrodes 112. However, after washing, the impedance of the electrodes 112 significantly increased on those yarns 111 that were not plasma treated before coating but remained the same on yarns 111 which were plasma treated.

Other than plasma 103, some embodiments wash and dry the fiber 111 before coating to remove spin finish oils or contaminants, etc. Additionally, or alternatively, some embodiments dry the fibers 111 using a drying over before coating to remove any water content in the fibers. This can improve the adhesion of the coating, as water may trap between the fibers and coating and cause faster degradation of the coating.

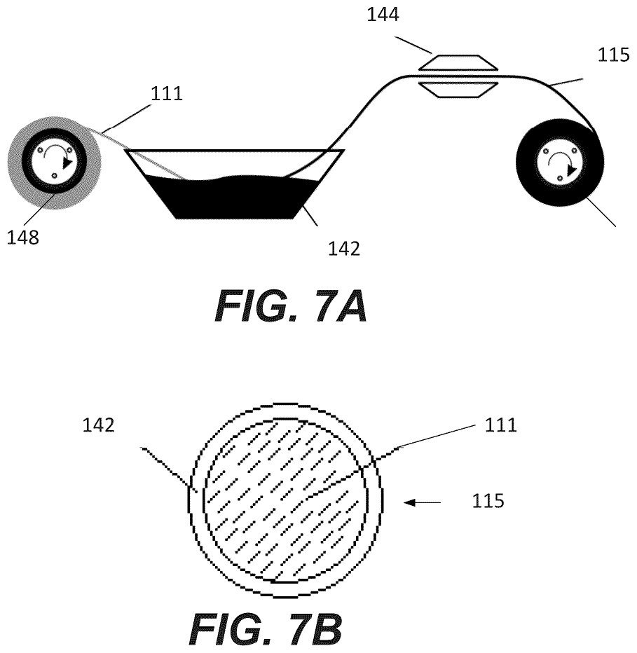

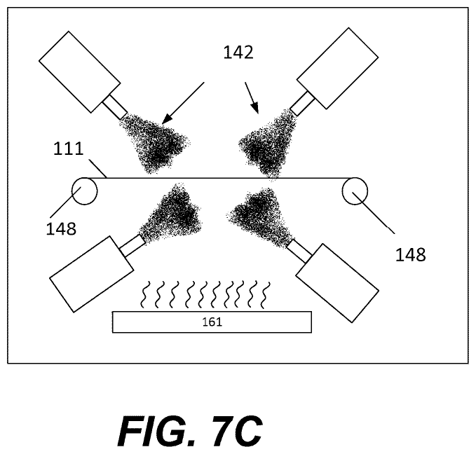

The process then proceeds to step 506, which applies conductive polymer fluid to each base fiber 111. FIG. 7A schematically shows a roll-to-roll coating process for applying a conductive polymer fluid to the base fiber 111 in accordance with illustrative embodiments. FIG. 7B schematically shows a cross-section of a coated base fiber 111 in accordance with illustrative embodiments. Various embodiments may refer to the application of conductive fluid as “coating” the base fiber 111. However, coating is not limited to merely contacting the outer surface. The term coating is intended to cover various embodiments including when the conductive fluid may penetrate and/or be partially absorbed into the fiber 111.

The conductive polymer fluid 142 is applied to each base fiber 111 individually and prior to assembly with other fibers 111. Among other things, the conductive polymer fluid 142 may include PEDOT:PSS, Polyaniline, Polypyrrole, and/or MXene. In various embodiments, the conductive polymer 142 may include one or more additives, e.g., to enhance conductivity or mechanical durability. For example, some embodiments may include additives to enhance conductivity, such as secondary dopants (e.g., polar solvents, surfactants, ionic liquids), and/or conductive particles (e.g., graphene, carbon black). Additionally, or alternatively, illustrative embodiments may include mechanical durability enhancements, such as elastomeric materials (e.g., polyurethane, PDMS), surfactants (e.g., zonyl, triton), plasticizers (e.g., glycerol), soft polymers, coated onto texturized yarns.

Various embodiments may include additives such as parylene, polyvinyl alcohol (PVA), poly(ethylene glycol)-block-poly(propylene glycol)-block-poly(ethylene glycol) (PEO20-PPO70-PEO20, Pluronic® P123), zonyl, glycidoxypropyltrimethoxysilane (GOPS), polyethylene glycol, divinyl sulfone (DVS) crosslinker, Ethylene glycol, and/or Dimethyl sulfoxide (DMSO) to improve the adhesion/mechanical/electrical properties.

The inventors discovered that applying the conductive polymer fluid 142 to individual fibers 111, rather than to a fabric of already assembled individual fibers 111, reduces cracking of the coating, provides better adherence and improves coverage of the surface area of fiber 111 by the conductive polymer fluid when applied.

In various embodiments, an unwound base yarn 111 is pulled moved through a conductive solution 142 by rollers 148. After the conductive solution 142 (e.g., coated, impregnated, etc.) is applied to the base fiber 111, the coated fiber 115 passes through a heating chamber 144 to help cure the conductive solution. The coated/impregnated fiber 115 is wound on the second roller 148 and is ready for assembly into a fabric portion 118. FIG. 7B schematically shows a cross-section of the coated/impregnated fiber 115. FIG. 7B is an example of a cross-section of the coated fiber 115. Illustrative embodiments are not limited to the cross-section shown or described herein. Furthermore, while some embodiments may have complete coverage of the outer surface of the base fiber 111 (and/or penetration into the interior of the single/multi strand fiber 115) and substantially uniform coating 142 thickness, some other embodiments may not.

FIG. 7C schematically shows an alternative process for applying a conductive polymer fluid to the base fiber 111 in accordance with illustrative embodiments. As shown, the base fiber 111 may be sprayed with the conductive fluid 142 by one or more spray nozzles. The conductive polymer fluid may be dispersed in a medium that allows for sufficient flowability to spray onto the fiber 111. In some embodiments, the spray chamber may include a heating element 161 that may be activated during or after the spraying of the conductive fluid 142.

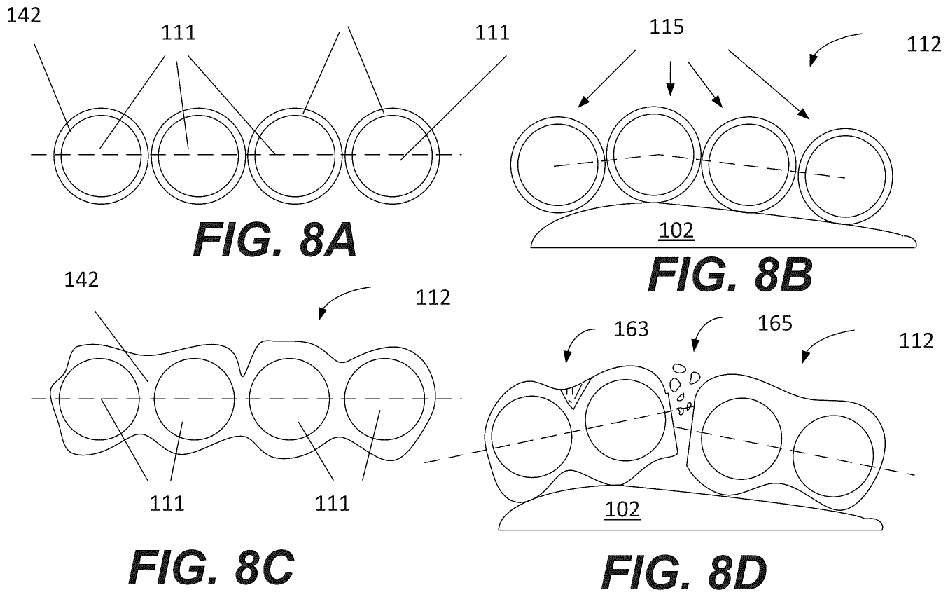

In addition to the methods shown and described with respect to FIGS. 7A and 8, a number of other methods may be used to apply the conductive fluid to the fiber 111 (e.g., to the exterior and/or interior of the fiber 111). For example, dip coating, knife-over-edge coating, slot-die coating, gravure coating, immersion coating, vapor deposition, spray coating, and/or sputtering may be used to coat the base fiber 111. After the base fiber 111 is coated/impregnated with conductive fluid it is referred to as the conductive fiber 115.

Returning to FIG. 5, the next step 508 in the process is to assemble the individually polymer coated fibers 115 into an ECG sensing electrode 112. For example, the individual conductive fibers 115 may be weaved, knitted, or embroidered (e.g., examples of interlacing) to form an ECG electrode 112, 114. The fibers 115 may be knitted, for example, using a Stoll flatbed knitting machine. In some embodiments, the conductive fibers 115 may be interspersed with non-conductive fibers 113, as shown in FIG. 4C. The ECG electrode 112 may be assembled in a circular or rectangular shape, among other shapes. In some embodiments, the ECG electrode 112 may include a shock absorber (e.g., a foam) and/or a support plate. As described previously, the shock absorber advantageously assists the ECG electrode 112 to achieve a more uniform pressure on the skin.

It is recognized that the term “coating” shown in the figures can refer to a coating on an exterior surface of the base fibers 111, can refer to an impregnation into an interior of the base fibers 111, or can refer to both a coating as well as to an impregnation, as desired. It is also recognized that the interior can refer to within a base material of a single stranded fiber 111. It is also recognized that the interior can refer to between the exterior surfaces of each strand in a multistranded fiber 111. It is also recognized that the interior can refer to both on the exterior surface of each strand as well as within the base material of each strand in a multistranded fiber 111. FIG. 8A schematically shows a plurality of individually coated/impregnated conductive fibers 115 assembled after application (e.g., coating) of the conductive polymer fluid. In various embodiments, because the base fibers 111 were coated/impregnated prior to assembly, the coating/impregnated solution 142 can be dispersed relatively evenly and consistently around/within the base fiber 111. In some embodiments, the base fiber 111 may be a multifiber base fiber 111 (e.g., multistrand), such as a yarn. In such embodiments, the multifiber 111 may be coated/impregnated prior to assembly with one or more other conductive fibers 115 (e.g., individually coated/impregnated fibers prior to their assembly as the electrode 112, 114 or otherwise assembled into the garment 105, 110 as described herein).

FIG. 8B schematically shows the plurality of individually coated/impregnated conductive fibers 115 of FIG. 8A in contact with the patient 102. The plurality of conductive fibers 115 may form at least part of the ECG electrode 112. As shown, the electrode 112 having individually coated/impregnated fibers 115 conforms well to the anatomy of the patient 102. This is because the fibers 115 are individually coated/impregnated, and therefore, are not adhered or fixed to one another. Accordingly, the fibers 115 may move relative to one another without impacting the structure of the coated/impregnated coating 142 (e.g., the cured or dried fluid). Thus, polymer-based ECG sensing electrodes are more flexible, and as such, better able to conform to the contours of the patient's 102 anatomy than conventional ECG electrodes that may be built from rigid metallic materials.

FIG. 8C schematically shows a plurality of coated/impregnated conductive fibers 115 assembled before coating/impregnating. Illustrative embodiments may coat/impregnate a plurality of base fibers 111 after they are assembled into a fabric portion 118. Advantages include a quicker coating/impregnation process (e.g., the coating 142 may be applied to the entire assembled (e.g., interlaced) fabric rather than individual base fibers 111). However, the inventors determined that coating/impregnating the already assembled fabric portion 118 (e.g., coating/impregnation post assembly of the fibers 111), as opposed to coating/impregnation the individual fibers 111 prior to assembly with one another to make the fabric portion 118/garment 105, 110, results in a worse quality ECG electrode 112. The inventors suspect that this is because bulk coating the already assembled fabric portion 118 causes the coating 142 to adhere a plurality of fibers 111 together, in other words to undesirably fill the spaces/voids with the conductive fluid polymer between the individual fibers 111, as shown by example.

FIG. 8D schematically shows the plurality of already assembled and then coated/impregnated conductive fibers 115 of FIG. 8C in contact with the patient 102. The coating/impregnation 142 becomes more susceptible to fragmenting 165 and/or cracking 163, thereby reducing the quality of the conductive coating/impregnation. The inventors have determined that such bulk coating/impregnation results in an ECG electrode 112 with a lower quality signal. Accordingly, as mentioned previously, the inventors discovered that applying (e.g., coating and/or impregnating) the conductive polymer fluid 142 to individual fibers 111 prior to assembly, rather than to a fabric of assembled individual fibers 111, reduces cracking of the coating, provides better adherence and improves coverage of the surface area of fiber 111. It is recognized that the polymer-based ECG sensing electrodes 112 are examples of the electrodes assembled using the individually coated/impregnated conductive fibers 115, as discussed herein.

Returning to FIG. 5, the process then proceeds to step 510, which couples the ECG electrode 112 with the smart garment 105,110. In various embodiments, the ECG electrode 112 may be integrated into the smart garment 105, 110. For example, as shown in FIGS. 4D-4E, the conductive fabric portion 118 may be stitched or otherwise joined to the garment 110.

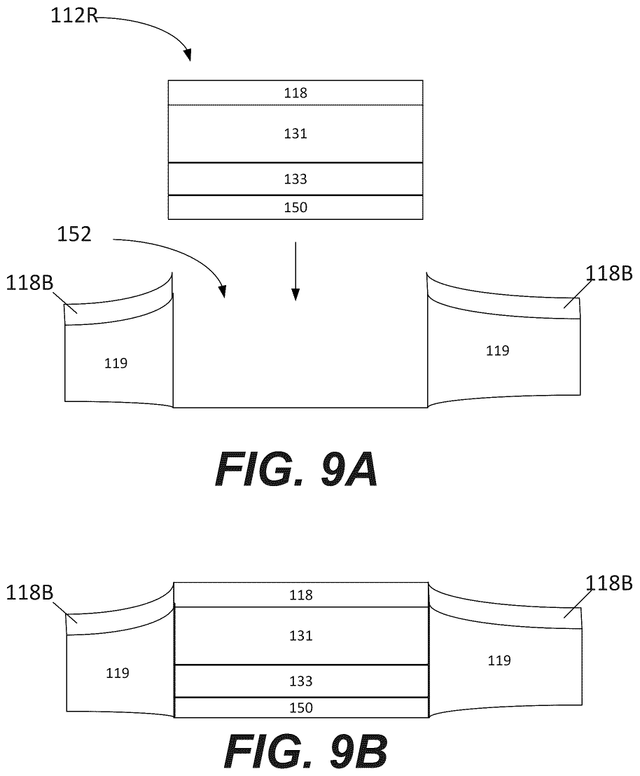

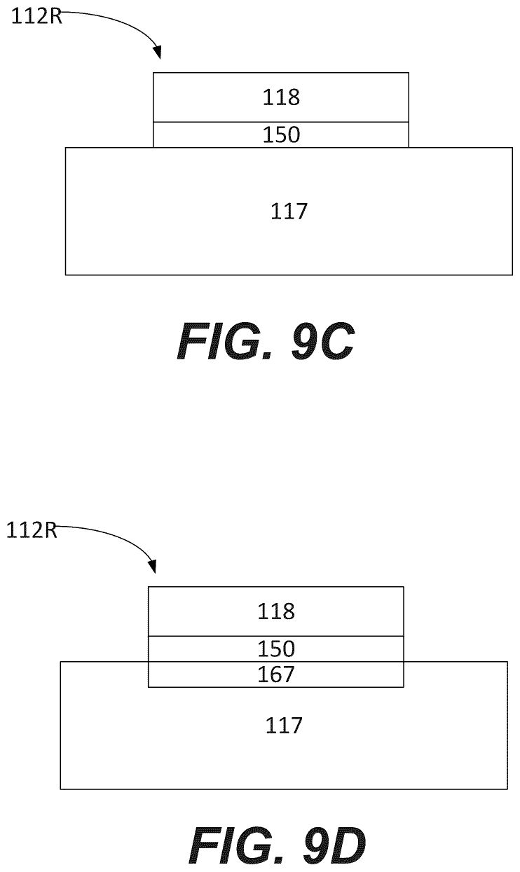

FIGS. 9A-9B schematically show a removable ECG electrode 112 in accordance with illustrative embodiments. The removable ECG electrode 112 may include a coupling portion 150, which may include hook and loop fasteners, snap connectors, and/or adhesive material. In various embodiments, the garment 110 may include a receiving portion 152 sized and positioned to receive the ECG electrode 112. FIGS. 9A-9B schematically show a removable electrode 112R being positioned into the receiving portion 152 of the smart garment 110. Although the shock absorber 131 and support panel 133 are shown, some embodiments may omit one or both of components. In some embodiments, the smart garment 110 may have integrated interconnects 118B. The receiving portion 152 may be configured so that the removable electrode 112R comes into electrical contact with the interconnect when it is seated in the receiving portion 152.

FIG. 9C schematically shows another embodiment of a removable ECG electrode 112R in accordance with illustrative embodiments. The removable ECG electrode 112R may be, for example, adhered to any surface of the smart garment 110 (e.g., by adhesive or hook and loop fasteners). Preferably, the top surface of the electrode 112R is positioned to contact the skin of the patient. FIG. 9D schematically shows another example of a removable ECG electrode 112R in accordance with illustrative embodiments. The removable ECG electrode 112R may be, for example, coupled to a counterpart coupling portion 167. In some embodiments, the coupling portion 150 and/or the counterpart coupling portion 167 may be magnetically attractive.

The process then proceeds to step 512, which positions the ECG sensing electrode 112 to receive patient ECG signals. To that end, the ECG electrodes 112 are preferably positioned on the skin of the user. In some embodiments, the smart garment may have receiving portions 152 in desirable locations. Additionally, or alternatively, the smart garment 110 may be configured so that the integrated ECG sensing electrodes 112 are positioned adjacent to desired skin positions of the user. When the user wears the smart garment 110, the ECG electrodes 112 desirably are positioned against the skin of the user 102 such that one or more of the electrodes 112 may receive ECG signals. In various embodiments, the polymer-based ECG sensing electrodes each have a signal-to-noise ratio of between 2.5 and 30.1 for the received ECG signals.

In examples, the polymer-based ECG sensing electrodes 112 are disposed in the smart garment 110 in locations/positions as described in further detail below. For example, a plurality of ECG leads are generated on polymer-based ECG sensing electrodes disposed on nonstandard or standard locations of the patient's body. For example, the nonstandard ECG signals include one or two ECG channels as follows.

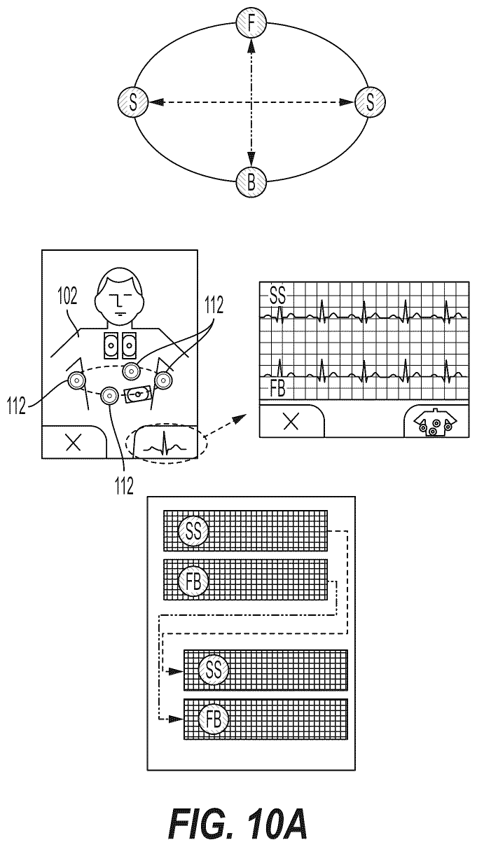

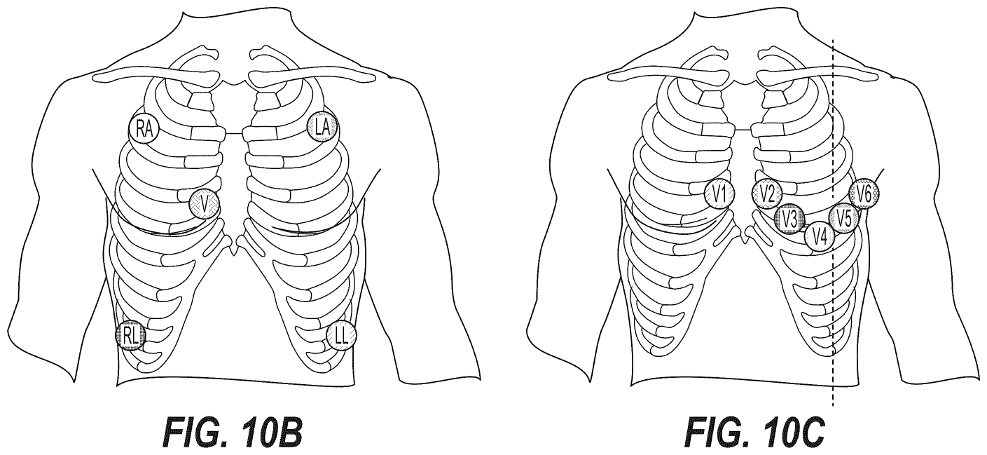

Referring briefly to FIG. 10A, there is shown at least four (4) polymer-based ECG sensing electrodes 112 disposed on the sides of the patient's 102 body and on an anterior and posterior position of the patient's 102 body. The polymer-based ECG sensing electrodes 112 are located over the rib cage, just under the breast area. For example, the polymer-based ECG sensing electrodes 112 can be positioned circumferentially at the level of the xiphoid process. In implementations, the polymer-based ECG sensing electrodes 112 are circumferentially placed along a transverse plane at the level of the xiphoid process. The left-side and right-side polymer-based ECG sensing electrodes 112 are positioned on the midaxillary line. The anterior ECG electrode 112 is positioned right of the sternum in the mid-clavicular line. The posterior ECG electrode 112 is positioned between about 4 cm to about 12 cm left of the center of the spine, more particularly between about 6 cm to about 10 cm left of the center of the spine, and more particularly about 8 cm left of the center of the spine. Accordingly, two non-standard ECG channels can include a side-side (SS) ECG channel, and a front-back (FB) ECG channel. Referring to FIG. 10A, in examples, the four (4) polymer-based ECG sensing electrodes are configured as two ECG leads that project onto a transverse plane an angle that is substantially orthogonal. In examples, the four (4) polymer-based ECG sensing electrodes 112 are configured as two ECG leads that project onto a transverse plane an angle of between 50 and 150 degrees. For example, wherein a first lead extends from a first geometrical center of a first one of the first pair of polymer-based ECG sensing electrodes 112 to a second geometrical center of a second pair of polymer-based ECG sensing electrodes 112. The second lead extends from a third geometrical center of a first one of the second pair of polymer-based ECG sensing electrodes 112 to a fourth geometrical center of a second one of the second pair of polymer-based ECG sensing electrodes 112. In this regard, projections of the first and second leads onto a transverse plane of the patient 102 comprises a substantially orthogonal angle. In this regard, projections of the first and second leads onto a transverse plane of the patient 102 comprises a substantially orthogonal angle. In some examples, projections of the first and second leads onto a coronal plane of the patient 102 comprises a substantially orthogonal angle. Together, the two ECG leads (channels) provide nonstandard ECG signals for analysis in accordance with the principles of the present disclosure.

Referring now to FIGS. 10B-10C, in examples, the polymer-based ECG sensing electrodes 112 are disposed in the smart garment 110 in standard locations/positions as described in further detail below. For example, the polymer-based ECG sensing electrodes 112 can be disposed within the smart garment 110 to generate standard ECG lead systems such as 3 lead systems, 5 lead systems, and 12 lead systems. For example, 3 lead system may use 3 polymer-based ECG sensing electrodes 112 located at RA, LA, and LL, and provide bipolar leads I, II, and III. For example, a standard 5 lead system may use 5 polymer-based ECG sensing electrodes 112 located at RA, RL, LA, LL, and chest location, and provide a bipolar leads I, II, and II, and a single unipolar lead depending on position of the chest polymer-based ECG sensing electrode 112 (e.g., positions V1-6). A standard 12 ECG leads can be generated by providing for polymer-based ECG sensing electrodes 112 in six limb leads, and six precordial leads (as noted above).

In various embodiments, the smart garment 110 may include a connector configured to couple with a controller such that the controller may receive the ECG signals. Illustrative embodiments may include such a connector, examples of which are described in copending patent applications PCT/CA2020/051789 and PCT/CA2020/051790, both of which are incorporated herein by reference in their entireties.

At step 514, the process determines that the patient 102 is having at least one arrhythmia episode. To make the determination, the ECG signals received by the one or more electrodes 112 may be sent to a controller for analysis. In order to detect an arrythmia, the patient is likely to have to wear the smart garment 110 for prolonged periods of time (i.e., to coincide with an arrhythmia incident). Preferably, the ECG electrode 112 is comfortable so as not to cause human skin irritation after predetermined test periods as set forth below (e.g., after 1 day of continuous contact exposure, after 2 days of continuous contact exposure, or after 3 days of continuous contact exposure). For instance, in some examples, the ECG electrode 112 is constructed so as to score zero or no more than one on the Human Skin Irritation Test set forth in Annex C of the ANSI/AAMI/ISO 10993-10:2010 standards for Biological Evaluation of Medical Devices—Part 10: Tests for Irritation and Skin Sensitization. Table C.1 of Annex C, which provides the grading scale for the Human Skin Irritation Test, is set forth below. In accordance with ISO 10993-10 C3.3, at least 30 volunteers shall complete the test, with no less than one-third of either sex. The ECG sensor 112 material shall be applied to intact skin at a suitable site, e.g., the upper outer arm. The application site shall be the same in all volunteers and shall be recorded. Generally, the ECG sensor 112 material shall measure at least 1.8 cm, preferably 2.5 cm in diameter. The ECG sensor 112 material shall be held in contact with the skin by means of a suitable non-irritating dressing, including non-irritating tape, for the duration of the exposure period. In one scenario, the ECG sensor 112 test material can be pre-moistened with water before application. To avoid unacceptably strong reactions, a cautious approach to testing shall be adopted. A sequential procedure permits the development of a positive, but not severe, irritant response. The ECG sensor 112 test materials are applied progressively starting with durations of 15 min and 30 min, and up to 1 h, 2 h, 3 h and 4 h. The 15 min and/or 30 min exposure periods may be omitted if there are sufficient indications that excessive reactions will not occur following the 1 h exposure. If no reaction or no excessive reactions are observed, the duration can be increased to 1 day, 2 days, and 3 days. Progression to longer exposures, including 24 h exposure at a new skin site, will depend upon the absence of skin irritation (evaluated up to at least 48 h) arising from the shorter exposures, in order to ensure that any delayed irritant reaction is adequately assessed.

Application of the material for a longer exposure period is always made to a previously untreated site. At the end of the exposure period, residual test material shall be removed, where practicable, using water or an appropriate solvent, without altering the existing response or the integrity of the epidermis. Treatment sites are examined for signs of irritation and the responses are scored immediately after mesh interface test material removal and at (1±0.1) h to (2±1) h, (24±2) h, (48±2) h and (72±2) h after patch removal. If necessary to determine reversibility of the response, the observation period may be extended beyond 72 h. In addition, the condition of the skin before and after the test shall be thoroughly described (e.g., pigmentation and extent of

TABLE C.1
Human skin irritation test, grading scale

| Description of response | Grading |
|---|---|
| No reaction | 0 |
| Weakly positive reaction (usually characterized by mild | 1 |
| erythema and/or dryness across most of the treatment site) | |
| Moderately positive reaction (usually distinct erythema or | 2 |
| dryness, possibly spreading beyond the treatment site) | |
| Strongly positive reaction (strong and often spreading | 3 |
| erythema with oedema and/or eschar formation) | |

hydration). Skin irritation is graded and recorded according to the grading given in Table C.1 of Annex C of ANSI/AAMI/ISO 10993-10:2010 standards for Biological Evaluation of Medical Devices—Part 10: Tests for Irritation and Skin Sensitization.

In various embodiments, the ECG electrodes 112 score a 0 or 1 grade on the human skin irritation test. Preferably, the ECG electrodes 112 score a 0 on the human skin irritation test.

After an arrythmia episode is detected for the patient, the process proceeds to step 516, which delivers a defibrillation shock to the patient via the therapy electrodes 114. For example, each defibrillation pulse can deliver between 25 and 400 joules of energy (e.g., between 60 to 180 joules) of energy. In some implementations, the defibrillating pulse can be a biphasic truncated exponential waveform, whereby the signal can switch between a positive and a negative portion (e.g., charge directions). This type of waveform can be effective at defibrillating patients at lower energy levels when compared to other types of defibrillation pulses (e.g., such as monophasic pulses). For example, an amplitude and a width of the two phases of the energy waveform can be automatically adjusted to deliver a precise energy amount (e.g., 150 joules) regardless of the patient's body impedance. A therapy delivery circuit can be configured to perform the switching and pulse delivery operations, e.g., under control of the processor 218. As the energy is delivered to the patient, the amount of energy being delivered can be tracked. For example, the amount of energy can be kept to a predetermined constant value even as the pulse waveform is dynamically controlled based on factors such as the patient's body impedance which the pulse is being delivered.

The process 500 then comes to an end. It should be noted that this process is substantially simplified from a longer process that normally would be used. Accordingly, the process may have many steps that those skilled in the art likely would use. In addition, some of the steps may be performed in a different order than that shown, or at the same time. Additionally, some of the steps above may be optional. Those skilled in the art therefore can modify the process as appropriate. For example, some embodiments may skip step 504. Additionally, in some embodiments some of the steps may occur substantially at the same time. For example, steps 508 and 510 may occur substantially at the same time. Some embodiments may assemble the integrated ECG electrode 112 into the smart garment 110, as the smart garment 110 is assembled.

It should be apparent that various embodiments are configured to be worn repeatedly and/or continuously. Accordingly, various embodiments provide a waterproof smart garment 110. In various embodiments, the ECG electrode 112 may also be waterproof. Furthermore, the ECG electrode 112 may be wash resistant (e.g., may be able to reliably detect ECG signals after a predetermined number of wash cycles). Advantageously, this allows the user to not have to remove and reapply the ECG electrodes 112 every time the smart garment 110 is washed. Additionally, the user does not have to worry about accidentally washing and destroying the ECG electrodes 112. The inventors believe that the methods of fabricating the ECG electrode 112 from conductive fibers 115 described herein enhance the useful lifespan of the ECG electrodes 112 and reduce susceptibility to delamination and cracking of the conductive coating, particularly after repetitive wash cycles.

In order to determine the long-term stability and longevity of coating on different substrates, each fiber material was coated under the optimized condition (150 uL/min, 190° C., and 20 rpm), then using a Stoll flatbed knitting machine the coated fibers were in a single jersey structure. Knitted fabrics and electrodes made of PEDOT:PSS-coated conductive fibers 115 were washed 30 and 60 times in accordance with the American Association of Textile Chemists and Colourists (AATCC) home laundry washing test method 61-2009, test No. 2A, using a SDLATLAS Launder-Ometer® (ROTOWASH, M228). After each laundering cycle, the samples were laid flat and left to dry at room temperature prior to the next wash cycle.

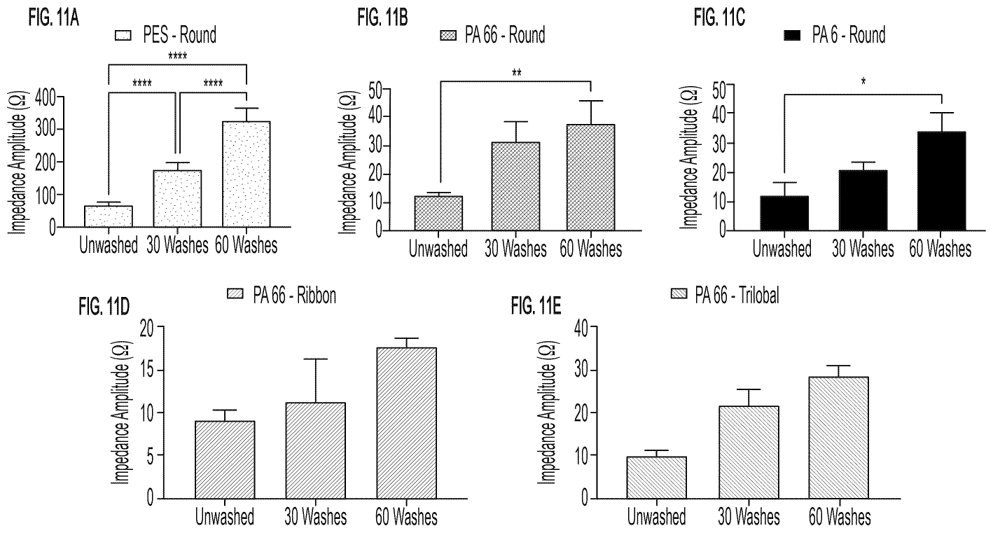

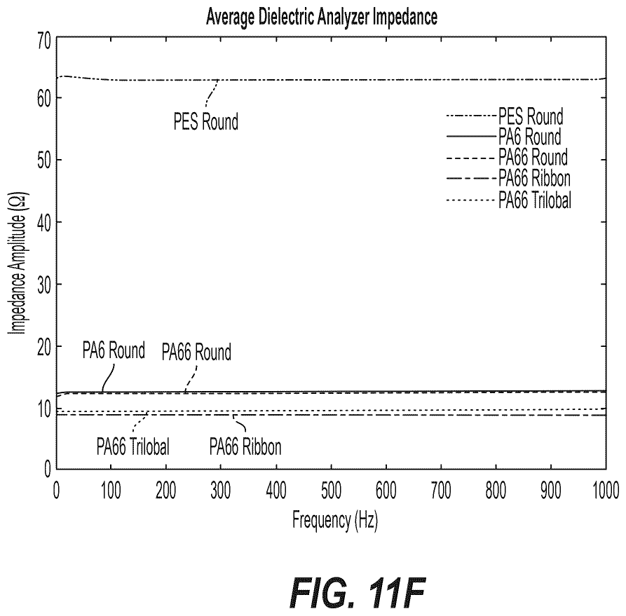

After laundering, samples were laid flat and left to dry overnight at room temperature prior to other measurements. A dielectric analyzer was used to measure the through-plane impedance of unwashed and washed fabrics. FIGS. 11A-11E show the through-plane impedance amplitudes of unwashed, 30× washed, and 60× washed knit fabrics made of PES-Round (FIG. 11a), PA 66—Round (FIG. 11b), PA 6—Round (FIG. 11c), PA 66—Ribbon (FIG. 11d), and PA 66—Trilobal (FIG. 11e) PEDOT:PSS-coated fibers at the frequency of 10 Hz. FIG. 11F schematically shows the average impedance amplitude for the fibers of FIGS. 11A-11E at a variety of different frequencies in accordance with illustrative embodiments. As shown, the pre-wash impedance values remain substantially constant at about 1 Hz, 10 Hz, 100 Hz, 1000 Hz, and ranges therebetween. For example, the impedance amplitude may be between about 8 Ohms and 12 Ohms at a variety of frequencies.

As shown in FIG. 11a, fabrics made of PES—Round coated fibers showed significant difference in through-plane impedance amplitudes between the unwashed, 30× washed, and 60× washed conditions (P value<0.0001). For fabrics made of coated PA6-Round and PA66-Round coated fibers, statistically significant difference in impedance amplitudes was observed between the unwashed and 60× washed samples (FIGS. 11b and 11c). For fabrics made of PA66-Ribbon and PA66-Trilobal coated fibers no statistically significant difference was observed in impedance for the wash conditions tested. The 60 times washed sample of PES—Round (FIG. 11a) showed the highest impedance amplitude of 324.5Ω among all the other samples (P value<0.0001). A relatively weaker adhesion of the PEDOT:PSS layer to the PES—Round fiber substrate might be the reason behind this increase in impedance over repetitive wash cycles. In contrast, the 60 times washed sample of PA 66-Ribbon (FIG. 11d) showed the lowest impedance amplitude of 17.6Ω among all the other samples. The PA 66-Ribbon fabric exhibited no observable change in electrical impedance property (FIG. 11d) and remained intact after 30 and 60 wash cycles demonstrating strong adhesion and superior stability of the PEDOT:PSS coating to the fiber substrate. Similarly, the coated PEDOT:PSS layer on the PA 66-Trilobal fiber substrate experienced minimal deterioration in electrical impedance property (FIG. 11e) after repetitive wash cycles (impedance amplitude of 28.28Ω for the 60-× washed sample). Accordingly, in various embodiments, the ECG electrode 112 is configured so that the impedance value that changes less than 150% after 30 wash cycles. In some embodiments, the ECG electrode 112 is configured so that the impedance value that changes less than 50% after 30 wash cycles. In various embodiments, the ECG electrode 112 is configured so that the impedance value that changes less than 150% after 60 wash cycles. In some embodiments, the ECG electrode 112 is configured so that the impedance value that changes less than 300% after 60 wash cycles.

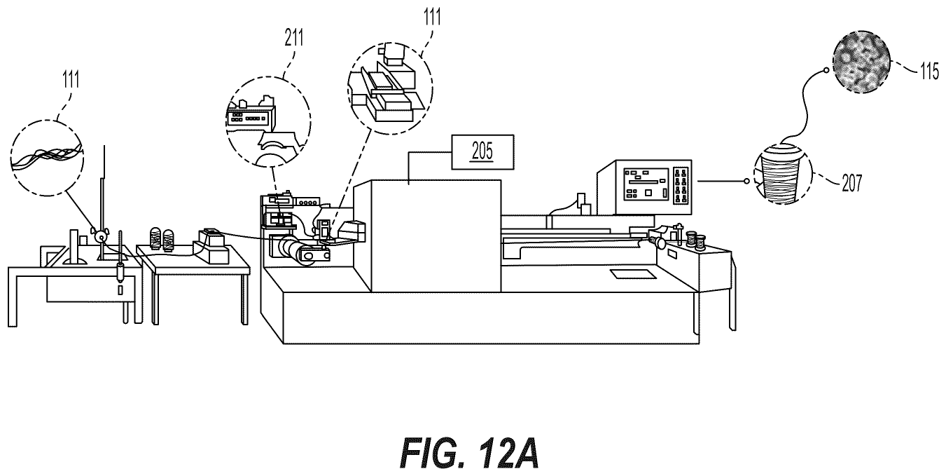

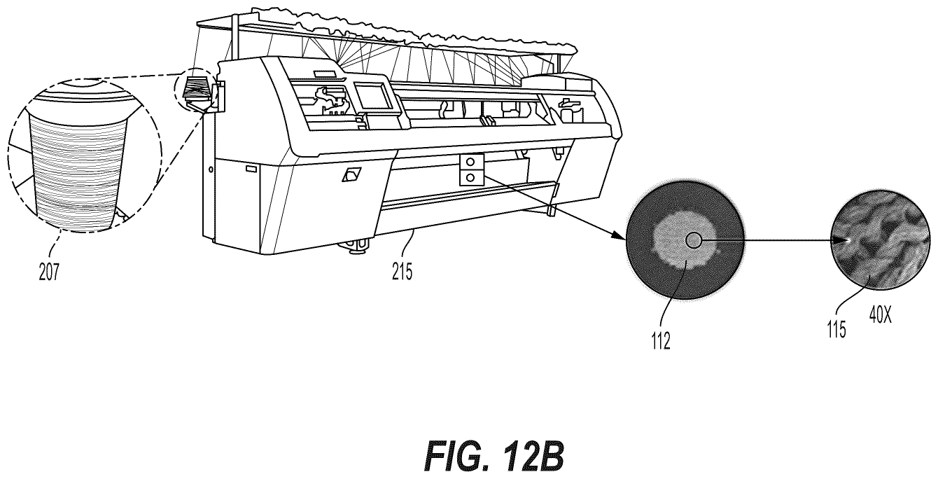

FIG. 12A shows a coating/impregnation system used to create conductive fibers 115 in accordance with illustrative embodiments. The system of FIG. 12A may be used as part of a roll-to-roll coating/impregnation method to create conductive fibers 115. For the sake of discussion, illustrative embodiments refer to coating the fibers with PEDOT:PSS. However, it should be understood that other embodiments may be coated/impregnated with another electrically conductive coating/impregnation. Also, as described previously, coating/impregnation is not limited to surface adhesion. In some embodiments, for example, the coating/impregnation may permeate into the pores of the fibers 111. Furthermore, in some embodiments, single fibers may be coated/impregnated and intertwined into a multifiber, such as a yarn. Additionally, or alternatively, the multifiber may also be coated/impregnated.

The system includes a base fiber 111 source, e.g., a spool of uncoated micro filaments. The uncoated base fiber 111 may pass under a syringe 211 having conductive fluid 142. The conductive fluid 142 contacts (e.g., is applied) the filament 111 and then passes into an oven 205. At the end, the coated/impregnated filament 115 is spooled onto a bobbin 207.

To investigate the effect of cross-sectional shape on coating/impregnation adhesion of the conductive ink 142 to fiber substrates 111, PA 66 fibers 111 with three different cross-sectional shape, i.e., circular, rectangular, and trilobal were tested. All the fibers 111 used in this study were provided by Myant Inc. (Canada). Table 1 below shows fiber 111 materials and their specifications:

TABLE 1

| | Fiber Cross-sectional | Polymer | Linear Mass Density |
|---|---|---|---|
| Sample Code | Shape | Materials | (Denier*) |
| PA 6-Round | Circular | PA 6 | 140 |
| PES-Round | Circular | PES | 140 |
| PA 66-Round | Circular | PA 6, 6 | 140 |
| PA 66-Ribbon | Ribbon/Flat | PA 6, 6 | 140 |
| PA 66-Trilobal | Trilobal | PA 6, 6 | 140 |

*Denier is the mass in grams per 9000 meters of a fiber which is a metric for the linear mass density of fibers.

Scanning Electron Microscopy (SEM) (JEOL, JSM 1000, Japan) was used to evaluate the morphological characteristics of fibers with different cross-sections (PA 66—Round, PA 66—Ribbon, and PA 66—Trilobal).

Response Surface Methodology (RSM) was applied in this study to determine desirable coating conditions. Preliminary experiments were performed to determine the range of coating/impregnation speed, coating rate and curing temperature before the experimental design. In this regard, experiments were carried out by varying a single factor while keeping all of the other factors constant. According to these experimental data, the total number of 15 experimental runs based on central composite design (CCD) were performed. Initial coating/impregnation rate (X1), curing temperature (X2), and coating speed (X3) with three levels (−1, 0, and 1) were the three factors in the coating process that were considered (Table 2). The test factors were coded based on Equation 1 in the regression equation:

$${Z}_{i} = \frac{{X}_{i} - {X}_{o}}{Δ X} \qquad Equation 1$$

where Zi is a dimensionless coded value of the i^{th} independent variable, Xᵢ is the actual value of independent variable, X₀ is the actual value of the independent variable at the center point level and ΔX is the step change of variable.

The response function (Yi, impedance (Ω)) is related to the coded variables using Equation 2:

$$Y = {b}_{0} + {\sum }_{i = 1}^{n} {b}_{i} {x}_{i} + {\sum }_{i = 1}^{n} {b}_{ii} {x}_{i}^{2} + {\sum }_{i = 1}^{n - 1} {\sum }_{j = i + 1}^{n} {b}_{ij} {x}_{i} {x}_{j} \qquad Equation 2$$

where Y is an observable response variable (impedance of electrode (Ω)), b0 is the constant coefficient, bi is the regression coefficients for linear effects, bii is the quadratic coefficients, bij is the interaction coefficients and xi, xj are the coded values of input factors.

Table 2 below shows experimental range and levels of independent variables for the coating/impregnation process:

TABLE 2

| | | | Range and Level | | |
|---|---|---|---|---|---|
| Variables | Factor | Unit | −1 | 0 | 1 |
| Coating Rate | X₁ | μL/min | 50 | 150 | 250 |
| Curing Temperature | X₂ | ° C. | 160 | 190 | 220 |
| Coating Speed | X₃ | rpm | 10 | 25 | 40 |

Although experimental rates for coating/impregnation speed were between 10 and 40 rpm, the inventors believe that various embodiments may coating/impregnation at lower speeds (e.g., of about 1 rpm). In terms of manufacturability, higher speeds are generally preferred, but lower speeds might be useful in special cases (e.g., where the length of the curing oven is shorter/not long enough).

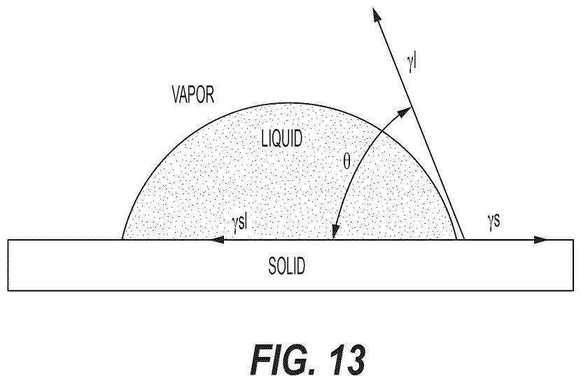

The contact angle measurement technique was applied in this study to investigate the surface physico-chemical properties of the base fibers 111. By measuring the contact angle θ formed at the intersection of the liquid, gas, and solid phases, a direct evaluation of the surface wettability of a solid substrate with a defined liquid can be provided. When a liquid 142 with a surface tension of γ_{L} is placed on a solid surface with a surface free energy of γ_{S}, the liquid will spontaneously form a droplet (FIG. 13) or spread on the surface (in case of complete wetting). FIG. 13 schematically shows a liquid drop showing the quantities of force balance at the interface in accordance with illustrative embodiments. In various embodiments, the conductive polymer fluid 142 has a surface tension of between about 30 mN/m and about 45 mN/m, or about 35 mN/m and about 40 mN/m, or about 39 mN/m. The conductive polymer fluid 142 may have a viscosity of between about 65 centipoise and about 85 centipoise, more particularly, about 70 centipoise to about 75 centipoise. In some embodiments, the conductive fluid 142 may have a viscosity in the range of about 0.5 cps to about 1000 cps, and surface tension range of 30-75 mN/m.

When a droplet is formed, a relationship between the solid surface free energy and the liquid surface tension can be established at equilibrium considering that the surface tensions of each phase are vectors acting at the edge of the drop. The interfacial tension between liquid and solid is named γ_{SL}. The thermodynamic equilibrium can be described by Young's equation (Equation 3):

$${γ}_{S} = {γ}_{SL} + {γ}_{L} Cos θ \qquad Equation 3$$

Where θ is the angle formed by the liquid drop on the solid surface. In this study, using different standard liquids listed in Table 3, the dispersive and polar components of the surface free energy (SFE) of each fiber was determined by the Owens, Wendt, Rabel and Kaelble (OWRK) method. The work of adhesin (W_{A}) which in this case is a measure of the strength of the physico-chemical interactions between the fiber and liquid was calculated from Equation 4 based on the surface free energy of the fiber (solid) and the surface tension of the conductive ink (liquid). The highest the W_{A} value, the stronger adhesion between the fibers and the ink.

$${W}_{A} = 2 {( {γ}_{S}^{d} {γ}_{L}^{d} )}^{0.5} + 2 {( {γ}_{S}^{p} {γ}_{L}^{p} )}^{0.5} \qquad Equation 4$$

the interfacial tension γ_{SL} between the fiber and a liquid, can be deduced from Dupré's equation (equation 5):

$${W}_{A} = {γ}_{S} + {γ}_{L} - {γ}_{SL} \qquad Equation 5$$

The lowest the interfacial tension value, the higher the longevity of adhesion between the fibers and the ink. Finally, combining Young's (Equation 3) and Dupré's (Equation 5) equations, the work of adhesion can be expresses as follow:

$${W}_{A} = {γ}_{L} ( 1 + Cos θ ) \qquad Equation 6$$

Based on the Young-Dupre equation (Equation 6), by direct measurement of the contact angle θ of a liquid/ink with a surface tension of γ_{L}, on a fiber substrate, the work of adhesion between the ink and the fibers can be determined.

Using K100 force tensiometer (KRÜSS Scientific), surface tension (σ) of the PEDOT:PSS conductive ink as a measure of work per unit area or force per wetted length was measured, as the conductive polymer fluid was applied. Using the plate method according to Wilhelmy, the force acting on an optimally wettable plate which is immersed vertically in the PEDOT:PSS conductive ink was measured. Initially, water was used as the sample liquid, and its surface tension was measured using the Wilhelmy plate method. This was used to verify the viability and accuracy of the instrument. To measure the surface tension of the conductive ink 142, a cleaned platinum plate was affixed into the K100. The surface of the ink solution was automatically determined by the instrument and the surface tension of the solution was measured. Minimum of three measurements were performed. A polytetrafluoroethylene (PTFE) substrate (2 cm×2.3 cm×0.1 cm) was used to determine the wetted length of the substrate. For contact angle measurements, the advancing and receding contact angles of the ink solution on PTFE substrate were measured where the substrate was immersed in and retracted out of the ink solution at a speed of 10 mm/min. The properties of the PEDOT:PSS conductive ink 142 can be seen in Table 3.

The Washburn method is based on the monitoring of capillary rise of a liquid in a tube filled with fibers. This method was used in this study to characterize the effect of fiber materials and geometrical cross-sections on the wettability of fibers. This method allows testing a large number of fibers in a single experiment thus giving a good statistical determination of the fiber wettability. Washburn's equation defines the flow of a liquid through a capillary tube:

$${h}^{2} ( t ) = \frac{r}{2} \frac{{γ}_{L} Cos {θ}_{a}}{η} t \qquad Equation 7$$

where h is the distance travelled by the liquid front, r is the radius of the capillary tube arrangement, θₐ is the advancing contact angle, η is the viscosity of the liquid and t is the flow time. The corresponding mass change over time can be deduced, it writes

$${m}^{2} ( t ) = \frac{{π}^{2} {r}^{5}}{2} \frac{{ρ}^{2} {γ}_{L} Cos {θ}_{a}}{η} t \qquad Equation 8$$

Following Washburn's modified approach, a porous material packed in a column can be described as a bundle of capillary tubes. Assuming that a mean capillary radius exists, referred to as r̅, one can derive a modified Washburn's equation for porous media

$${h}^{2} ( t ) = [ \frac{( c \overline{r} )}{2} ] \frac{{γ}_{L} Cos {θ}_{a}}{η} t \qquad Equation 9$$

where c is a constant accounting for the tortuous path of the liquid in the equivalent capillary tubes arrangement. Then a tensiometer, here a K100 tensiometer, can be used to track the rise of the liquid by recording the weight of liquid penetrating the cylindrical porous medium

$${m}^{2} ( t ) = [ \frac{( c \overline{r} ) {{ϵ}^{2} ( π {R}^{2} )}^{2}}{2} ] \frac{{ρ}^{2} {γ}_{L} Cos {θ}_{a}}{η} t \qquad Equation 10$$

$$C = [ \frac{( c \overline{r} ) {{ϵ}^{2} ( π {R}^{2} )}^{2}}{2} ] \qquad Equation 11$$

$${m}^{2} ( t ) = C \frac{{ρ}^{2} {γ}_{L} Cos {θ}_{a}}{η} t \qquad Equation 12$$

where ϵ is the relative porosity and R is the inner radius of the measuring tube. Knowing the mass variation over time m(t), the apparent wetting properties of the porous medium are derived from Equation 12. In this study, the sample holder used for the measurements consists of a hollow cylinder in which the fibers were placed. A piston ensures the compaction of the fiber arrangement, and equivalently the global fiber volume fraction from which the relative porosity (Equation 10) can be calculated.

The dynamic contact angle measurement, according to the modified Washburn's equation, consists of two tests: the first one to set constant C, and the second one for the contact angle calculation with a test liquid. For the first test, in order to get rid of the contact angle in the modified Washburn's equation (Equation 12) it is necessary to use a totally wetting liquid (such as n-hexane) which has an apparent contact angle of 0° with the porous medium due to its low surface tension (Table 3). Then, by plotting the square of the mass gain m² as a function of time, and through a linear fit of the liquid intake stage, the geometric porous medium constant C can be determined knowing liquid density, surface tension and viscosity. Once constant C is determined, it may be substituted into the modified Washburn's equation (Equation 12), and the advancing contact angle can be calculated through a series of tests with other liquids (DI water, diiodomethane, and ethylene glycol were used).

In this study, a tube with an inner diameter of 1.6 cm diameter, was filled with 0.5 g of each fiber material. The loaded tube was affixed into the K100 vertically. A vessel containing the wicking liquid was raised under the tube until the bottom of the tube was immersed in the liquid. The liquid wicked up into the fiber bundle and the weight gain of the tube was recorded as a function of time. All tests were carried out under standardized conditions at 22° C. The properties of liquids including their density, viscosity, and surface tension used in these measurements are given in Table 3. A practical limitation in that the wicking liquid must be relatively low in viscosity so that the tests can be completed in a reasonable length of time. In some embodiments, loading the fiber into the tube is only suitable when the fibers are strong enough to double over for loading into the tube.

TABLE 3
Liquid properties and specifications

| | Density | Viscosity | Surface tension | |
|---|---|---|---|---|
| Liquid | ρ [g/ml] | η [mPas] | σ [mN/m] | Supplier |
| Deionized water | 0.997 | 0.9321 | 72.3 | Alphachem |
| n-Hexane | 0.6594 | 0.302 | 18.4 | Sigma-Aldrich |
| Diiodomethane | 3.2992 | 2.67 | 50 | Sigma-Aldrich |
| Ethylene glycol | 1.1088 | 17.5 | 47.5 | Sigma-Aldrich |
| PEDOT:PSS Ink | 1.012 | 70.84 | 45.2 | Myant Inc. |

All PEDOT:PSS-coated/impregnated fibers 115 developed in this study were successfully knitted into single jersey fabrics (for the through-plane impedance measurements) and textile electrodes using an industrial scale V-bed flat knitting machine 215 (Stoll, Ruetlingen, Germany). As shown in FIG. 12B, the dry textile electrode 112 design in this work has a 3D structure to ensure proper skin-electrode contact is established and maintained. To create this 3D structure, three layers were knitted and seamlessly integrated: 1) the surface of the electrode was made of PEDOT:PSS-coated fibers, 2) the spacer layer was knitted under the surface layer from polyester yarn acting as a filler and creating a 3D raised structure, and 3) the back layer knitted with polyester yarn to provide support to the entire structure. In order to create the spacer layer behind the electrode, the tuck stitch operation was applied. In this process, the non-conductive polyester yarns that are knitting the technical back could tuck and float behind the coated/impregnated fiber 115 to create a spacer layer and ultimately a 3D pattern. Knitted electrodes 112, 114 were circular in shape with a 2 cm diameter (FIG. 12B). The surrounding fabric of the electrode 112, 114 had a double jersey structure made of polyester/spandex yarns (OMTEX/Invista) in both front and back layers.

Knitted fabrics and electrodes 112, 114 made of individually PEDOT:PSS-coated/impregnated fibers 115 (i.e. fibers 115 coated/impregnated prior to their assembly/interlacing into the electrode 112, 114) were washed 30 and 60 times according to the American Association of Textile Chemists and Colourists (AATCC) home laundry washing test method 61-2009, test No. 2A, using a SDLATLAS Launder-Ometer® (ROTOWASH, M228). After each laundering cycle, the samples were laid flat and left to dry at room temperature prior to the next wash cycle.

The through-plane electrical impedance of single jersey knit fabric samples made of PEDOT:PSS-coated fibers 115 were measured using an Alpha-A high performance dielectric impedance analyzer (Novocontrol Technologies GmbH & Co. KG) at a voltage of 1.0 V. The electrical properties of samples were analyzed across frequencies ranging from 0.1 to 10000 Hz. The electrical impedance at a frequency of 10 Hz was reported in this study.

The phantom skin recipe used in this study was obtained from literature. For each test, a phantom model (18 cm×13 cm×1 cm) was produced. The developed phantom had the most consistent and reproducible electrical properties that simulated those of human skin. Deionized water, agar (ThermoFisher SCIENTIFIC), ultra-high molecular weight polyethylene powder (125 μm average particle size, SigmaAldrich), sodium chloride (NaCl, ALPHACHEM), TX-151 (OIL CENTER RESEARCH INTERNATIONAL L.L.C., USA) and sodium azide (NaN3, BioShop, Canada) were the ingredients used. 1 g of NaCl and 0.05 g of NaN3 were dissolved in 85.82 ml of boiling water followed by gradually adding 2.62 g of the Agar. After heating the mixture for 20 minutes, the mixture was removed from heat and small quantities of TX-151 (2.07 g in total) were sprinkled into the mixture several times and quickly mixed to assure a homogenous mixture. Lastly, the polyethylene (8.44 g in total) was gradually added to the solution and mixed well to break up any clumps and ensure a uniform consistency. Once all ingredients were added, the mixture was poured into a container, which must be immediately closed (with a plastic lid) to prevent evaporation. The phantom was covered, and the mixture was allowed to completely cool down to room temperature (for about a day) to completely solidify.

Electrochemical impedance spectroscopy (EIS) of electrodes was measured using an Ivium Vertex One potentiostat (Ivium Technologies, Eindhoven, Netherlands) over the frequency range of 1 to 10000 Hz with an applied constant current of 1 mA. Swatches were knitted containing electrodes with each of the described PEDOT:PSS-coated fibers (n=5/material type). EIS measurements were configured with three electrode systems: dry electrodes made of PEDOT:PSS-coated fibers were used as working electrode, and Ag/AgCl electrodes (Kendall™ 100 Series Foam Electrodes, Covidien, MA, USA) were used as counter and reference electrodes. The electrodes were placed 1.5 cm apart on the phantom. The spacing between the electrodes was kept consistent on all measurements to reduce spatial variability. To eliminate electrode surface area as a variable and for the scope of this experimental portion, all electrodes used in this study had the same surface area (3.14 cm²) and all phantom samples were the same size. EIS measurements were completed directly on the smooth surface of the phantom. To reduce environmental variables, EIS testing was completed in a controlled lab at 22° C. and 52% relative humidity. New Ag/AgCl electrodes were used for every EIS measurement.

Skin-electrode impedance measurements were performed using the measurement protocols described by Spach et al. Galvanostatic electrical impedance spectroscopy measurements were done over 1 Hz to 10 KHz frequency range (3 points/decade) with a current range between 100 nA and 100 μA, using a PalmSens4 (PalmSens BV, Houten, Netherlands). Swatches were knitted containing electrodes with each of the described PEDOT:PSS-coated fibers (n=5/material type). Knitted swatch electrodes 112 were circular with a 2 cm diameter (FIG. 12B). Impedance measurements were also done on gel adhesive electrodes as the gold standard electrodes for electrophysiological recordings. The impedance measurements were obtained from one participant with five samples per fiber type.

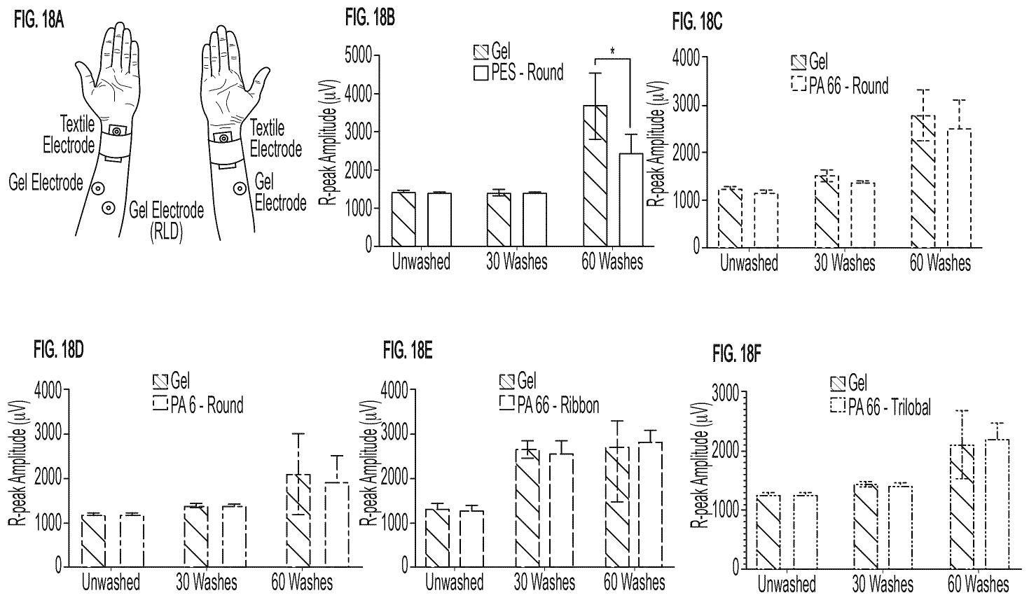

In order to compare and validate the recording fidelity and performance of textile electrodes, simultaneous measurements were also done with gel adhesive electrodes. FIG. 18A shows the placement of textile and gel electrodes on the subject's forearms. Recordings were done when the subject was sitting, at rest. Textile electrodes were fixed onto the skin using adjustable straps around the forearm (FIG. 18A). The pressure between the dry textile electrodes and the skin (applied by the straps) was controlled by calibrated pressure measurements at the time of their placement. The targeted skin-electrode pressure was 20 mmHg. ECG recordings were done simultaneously from the gel and textile electrode pairs using an 8-channel OpenBCI Cyton biosensing system (OpenBCI company, Brooklyn, USA) at a 250 Hz sampling frequency. All analyses were performed using Python (Ver. 3.8.8). Before finding R-peak locations, the signals were filtered using a fourth order Butterworth bandpass filter 0.05 Hz-125 Hz. To remove outliers, any R-peak-to-peak amplitude larger than 5000 uV was eliminated. This value is much higher than typical R-peak amplitudes at wrist position. Amplitudes that were detected higher than 5000 uV are likely to be motion artifacts that were falsely detected as R-peaks.

GraphPad Prism software (Ver 8, GraphPad Software Inc, San Diego, USA) was used to assess the statistical significance of all comparison studies in this work. Sample size of n=5 was selected otherwise mentioned in specific experiments. In the statistical analysis for group comparisons between multiple samples, two-way ANOVA followed by Tukey's multiple comparisons tests were performed with the alpha value of 0.05. For comparisons between washed and unwashed groups, t-tests were used. In all statistical tests, the significant level was 0.05. The bars in each of the statistical analysis plots represent the mean, and the error bars represent the standard error of the mean. If any significant differences are found, the ‘*’ is used (p<0.05 for all plots).

The cross-sectional images of PA 66—Trilobal, PA 66—Round, and PA 66—Ribbon, are shown in FIGS. 14A-14F. To observe the cross-sectional shape of PA 66 fibers, the fibers were cut using the microtome technique.

FIGS. 14A-14F shows a picture of cross sections of PA 66 fibers, particularly: (14a) trilobal at 1000 magnification, (14b) trilobal at 500 magnification, (14c) circular at 1000 magnification, (14d) circular at 500 magnification, (14e) ribbon at 1000 magnification, (14f) ribbon at 500 magnification in accordance with illustrative embodiments.

Under favorable conditions, surface forces cause liquid to be wicked into a bundle of fiber. The work of adhesin (W_{A}) represents the strength of the physico-chemical interactions between the fiber and PEDOT:PSS ink which was calculated based on the OWRK method using Washburn's technique for experimental measurement. The W_{A} and γ_{L} values that are reported in Table 4 represents the work of adhesion and interfacial tension between the PEDOT:PSS conducting ink and various fiber substrates that were used in this study. PA 66 fiber with a Ribbon cross-sectional shape had the highest the W_{A} value, which is an indication of a stronger adhesion between the fibers and the ink. The same material (PA 66—Ribbon) also had the lowest interfacial tension value, which represents a higher longevity of adhesion between the fibers and the ink. Based on these results of these measurements, among all the other fiber materials, the PA 66—Ribbon material is expected to have the best performance in terms of durability of the coating layer after repetitive wash cycles due to the stronger adhesion of the conducting ink to the fiber substrate. The free energy of wetting results can be converted to contact angle values by the use of Young's equation (Equation 3). From the nature of the wicking process, it is apparent that these are “advancing” contact angles. It is obvious from the data in Table 4 that PEDOT:PSS ink is supposed to wet the PA 66—Ribbon fibers completely. However, the contact angle of the conducting ink on PES—round fibers is calculated to be around 43° which represents incomplete wetting of the fiber by the ink.

TABLE 4

| | Work of Adhesion | Interfacial Tension | Contact Angel |
|---|---|---|---|
| Fiber | W_{A} [mN/m] | γ_{SL}[mN/m] | θ [°] |
| PES-Round | 68.20 ± 1.70 | 0.85 ± 0.02 | 43.75 ± 3.57 |
| PA 6-Round | 74.71 ± 1.98 | 0.59 ± 0.01 | 27.55 ± 6.45 |
| PA 66-Round | 74.51 ± 2.05 | 0.26 ± 0.01 | 28.15 ± 6.09 |
| PA 66-Ribbon | 80.36 ± 1.86 | 0.19 ± 0.01 | 0 ± 3.00 |
| PA 66-Trilobal | 72.06 ± 2.08 | 0.36 ± 0.01 | 29.09 ± 5.29 |

Adhesion properties of PEDOT:PSS conducting ink to fiber substrates

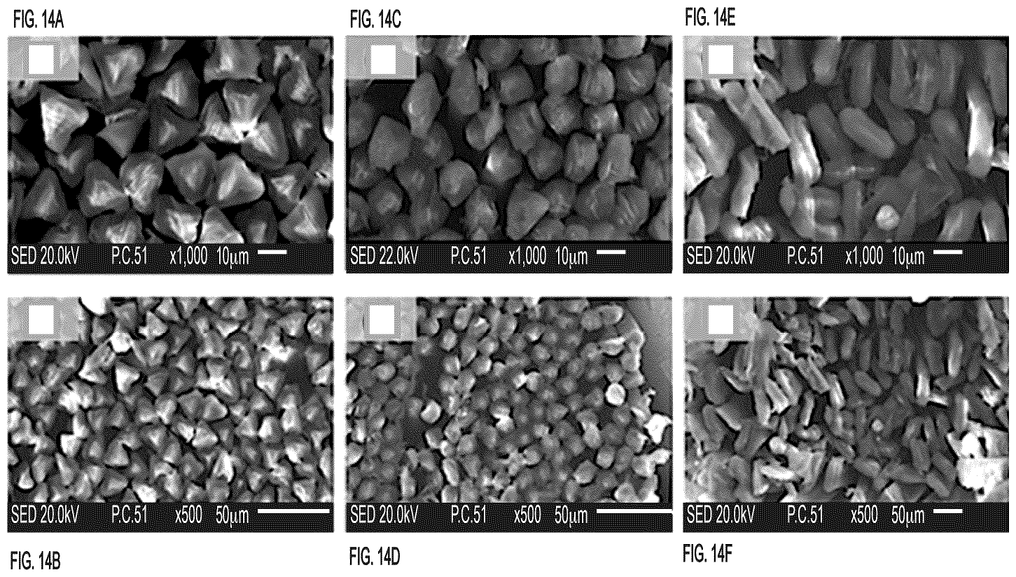

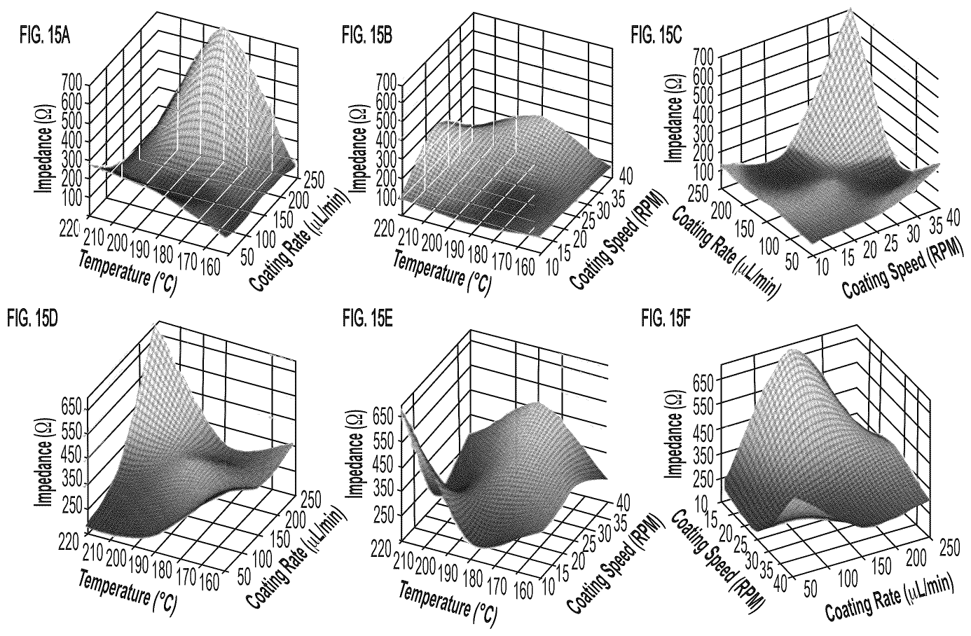

Results are shown as surface plots of unwashed and 60× washed electrodes made of PEDOT:PSS-coated PES fibers that can be seen in FIGS. 15A-15F. As shown in FIGS. 15b, 15c, 15e, and 15f, the electrode impedance amplitude on skin model increased at higher coating speed values, but by decreasing the coating speed beyond a specific value, the increase in impedance was not significant. Increasing the coating rate will increase the linear density of the ink (μL/m), therefore the ink may need more time to cure or flow into the interstices of the fiber substrate (FIGS. 15a, 15c, 15d, 15f).

Consequently, at low curing temperatures or high coating speed, increasing coating rate will cause mechanical degradation, this is more pronounced in impedance amplitude of 60× washed samples (FIGS. 15d, 15f). Based on the value of impedance amplitude after repetitive wash cycles, the coating condition was chosen based on the sections of the surface plots where the lowest impedance amplitude could be achieved. Therefore, coating rate of 150 μL/min, curing temperature of 190° C., and coating speed of 20 rpm were selected as the coating condition and used in the rest of this study to coat the fibers with the PEDOT:PSS ink.

In order to determine the long-term stability and longevity of coating on different substrates, each fiber material was coated under the optimized condition (150 μL/min, 190° C., and 20 rpm), then using a Stoll flatbed knitting machine the coated fibers were in a single jersey structure. The knitted fabrics were washed 30 and 60 times according to AATCC 61:2009. After laundering, samples were laid flat and left to dry overnight at room temperature prior to other measurements. A dielectric analyzer was used to measure the through-plane impedance of unwashed and washed fabrics. FIGS. 11A-11E shows the through-plane impedance amplitudes of unwashed, 30× washed, and 60× washed knit fabrics made of PES—Round (FIG. 11a), PA 66—Round (FIG. 11b), PA 6—Round (FIG. 11c), PA 66—Ribbon (FIG. 11d), and PA 66—Trilobal (FIG. 11e) PEDOT:PSS-coated fibers at the frequency of 10 Hz. As shown in FIG. 11a, fabrics made of PES—Round coated fibers showed significant difference in impedance amplitudes between the unwashed, 30× washed, and 60× washed conditions (P value<0.0001). For fabrics made of coated PA6—Round and PA66—Round coated fibers, statistically significant difference in impedance amplitudes was observed between the unwashed and 60× washed samples (FIGS. 11b and 11c). For fabrics made of PA66—Ribbon and PA66—Trilobal coated fibers no statistically significant difference was observed in impedance for the wash conditions tested. The 60 times washed sample of PES—Round (FIG. 11a) showed the highest impedance amplitude of 324.5Ω among all the other samples (P value<0.0001). A relatively weaker adhesion of the PEDOT:PSS layer to the PES—Round fiber substrate might be the reason behind this increase in impedance over repetitive wash cycles. In contrast, the 60 times washed sample of PA 66—Ribbon (FIG. 11d) showed the lowest impedance amplitude of 17.6Ω among all the other samples. The PA 66-Ribbon fabric exhibited no observable change in electrical impedance property (FIG. 11d) and remained intact after 30 and 60 wash cycles demonstrating strong adhesion and superior stability of the PEDOT:PSS coating to the fiber substrate. Similarly, the coated PEDOT:PSS layer on the PA 66—Trilobal fiber substrate experienced minimal deterioration in electrical impedance property (FIG. 11e) after repetitive wash cycles (impedance amplitude of 28.28Ω for the 60× washed sample).

FIGS. 11A-11E show the through-plane impedance amplitude of unwashed, 30× washed, and 60× washed fabrics made of PEDOT:PSS-coated fibers at frequency of 10 Hz in accordance with illustrative embodiments. 11a) Impedance amplitude of unwashed, 30× washed, and 60× washed fabrics of coated PES—Round fibers. 11b) Impedance amplitude of unwashed, 30× washed, and 60× washed fabrics of coated PA 66—Round fibers. 11c) Impedance amplitude of unwashed, 30× washed, and 60× washed fabrics of coated PA 6—Round fibers. 11d) Impedance amplitude of unwashed, 30× washed, and 60× washed fabrics of coated PA 66—Ribbon fibers. 11e) Impedance amplitude of unwashed, 30× washed, and 60× washed fabrics of coated PA 66-Trilobal fibers. n=10 fabric samples per fiber material. Bars represent the mean and error bars are the standard error of the mean. ‘*’ symbol represents p<0.05.

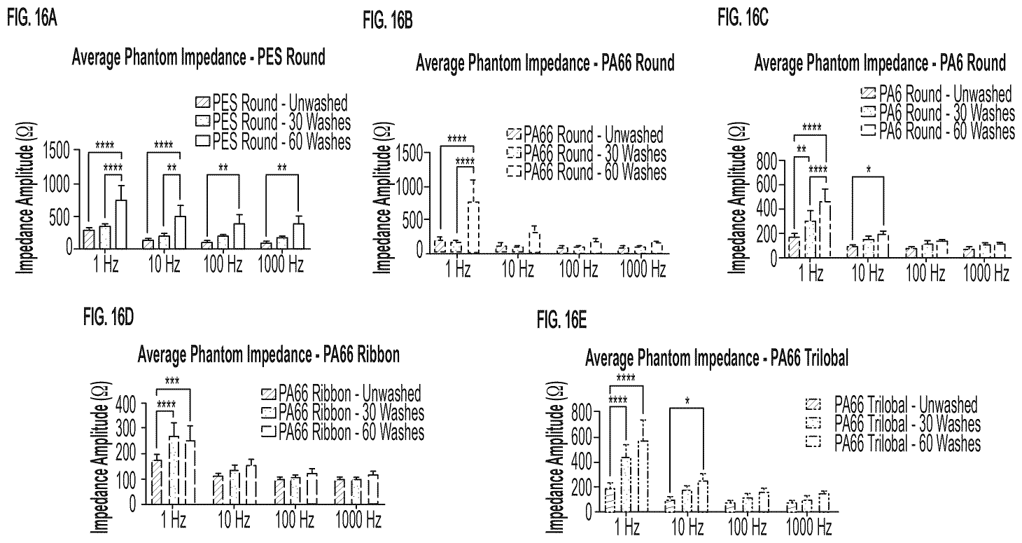

While various types of PEDOT:PSS coatings has been intensively studied to improve electrical properties of bioelectrodes (e.g., low impedance, low rigidity, and high charge injection capacity), one major hurdle that significantly limits the utility of PEDOT:PSS-coated electrodes in practical applications is their poor stability. To ensure long-term functionality and reliability of electrode materials in physiological environment, especially after repetitive wash cycles, the electrochemical impedance spectroscopy (EIS) measurements of knit electrodes on phantoms were performed (see FIGS. 16A-16E). The EIS bar graphs show the impedance amplitude of unwashed, 30× washed, and 60× washed electrodes of each coated fiber at frequencies of 1 Hz, 10 Hz, 100 Hz, and 1000 HZ. Based on the bar graphs in FIGS. 16A-16E, it can be seen that the PEDOT:PSS-coated PA 66—Ribbon fibers had lower electrical impedance amplitudes among all the other electrode samples made of different materials. For PEDOT:PSS-coated PES—Round electrodes, at 1 Hz and 10 Hz, statistically significant difference was found for samples of 60× washed in comparison to samples of unwashed and 30× washed (p-values of <0.0001). For electrodes of PES—Round at 100 Hz and 1000 Hz, statistically significant differences were found between unwashed samples and samples of 60 washes (p-values of 0.0018 and 0.0041, respectively). For PEDOT:PSS-coated PA66—Round electrodes at 1 Hz, statistically significant difference was found for samples of 60× washed in comparison to samples of unwashed and 30× washed (p-values of <0.0001). For PEDOT:PSS-coated PA 6—Round electrodes at 1 Hz, statistically significant differences were found between samples of unwashed and 30× washed, samples of unwashed and 60× washed, and samples of 30× washed and 60× washed (p-values of 0.0014, <0.0001 and <0.0001, respectively). For the same material at 10 Hz, statistically significant differences were found between samples of unwashed and 60× washed (p-values of 0.0292). For PEDOT:PSS-coated PA 66—Ribbon electrodes at 1 Hz, statistically significant differences were found for unwashed samples in comparison to samples of 30× washed and 60× washed electrodes (p-values of <0.0001 and 0.001). For PEDOT:PSS-coated PA 66—Trilobal at 1 Hz, statistically significant differences were found for unwashed samples in comparison to samples of 30× washed and 60× washed (p-values of <0.0001). Statistically significant differences were found between the unwashed and 60× washed PA 66—Trilobal electrodes at 10 Hz (p-values of 0.0119). No statistically significant differences were found for The rest of the samples of different electrode materials between washability conditions.

FIGS. 16A-16E show average impedance amplitude of unwashed, 30× washed, and 60× washed electrodes at 1 Hz, 10 Hz, 100 Hz, and 1000 Hz in accordance with illustrative embodiments, a) electrodes made of PEDOT:PSS-coated PES—Round fibers. b) electrodes made of PEDOT:PSS-coated PA 66—Round fibers. c) electrodes made of PEDOT:PSS-coated PA 6—Round fibers. d) electrodes made of PEDOT:PSS-coated PA 66—Ribbon fibers. e) electrodes made of PEDOT:PSS-coated PA 66—Trilobal fibers. n=5 electrodes per fiber material. Bars represent the mean and error bars are the standard error of the mean. ‘*’ symbol represents p<0.05.

To further investigate the effect of the repetitive wash cycles on electrical property of the coated layer, electrochemical impedance spectroscopy (EIS) analysis of the 3D knit electrodes made of PEDOT:PSS coated fibers was conducted on phantom skin models.

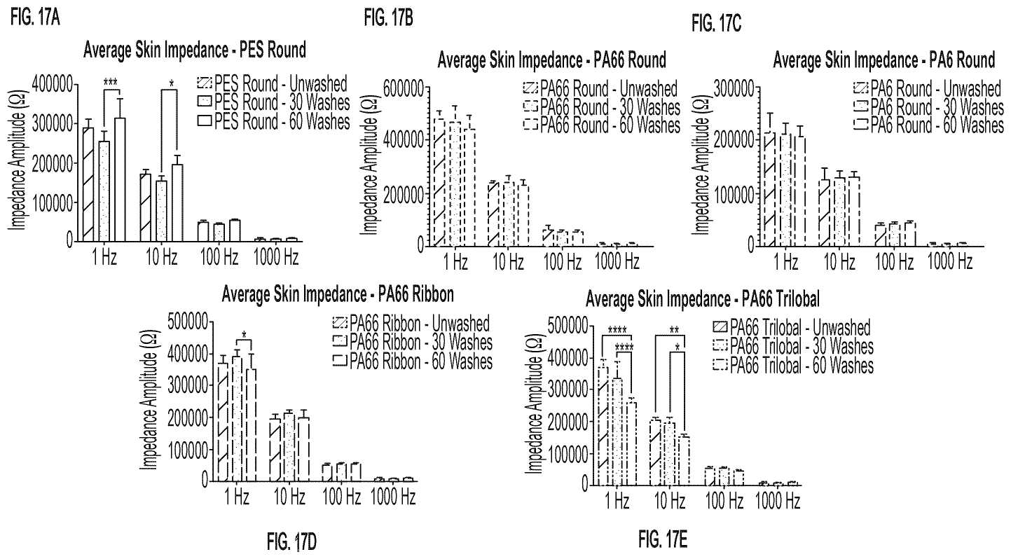

In order to assess the electrical properties of knitted textile electrodes made of PEDOT:PSS-coated fibers developed in this study, electrode-skin impedance measurements were performed. Electrode-skin impedance measurement results are illustrated as bar graphs in FIGS. 17A-17E, showing the impedance amplitudes of unwashed, 30× washed, and 60× washed textile electrodes over the frequency range of 1 Hz to 1000 Hz. At frequencies of 1 Hz and 10 Hz, there is a statistical difference between 30 and 60 times washed PES—Round electrodes whereas no statistical difference was observed between the unwashed, 30× washed, and 60× washed electrodes of other materials. As the frequency increases, the differences in impedance amplitude of unwashed, 30×, and 60× washed samples of each material fluctuates and decreases.

FIGS. 17A-17E shows Average electrode-skin impedance amplitude of unwashed, 30× washed, and 60× washed electrodes at 1 Hz, 10 Hz, 100 Hz, and 1000 Hz in accordance with illustrative embodiments, a) electrodes made of PEDOT:PSS-coated PES—Round fibers. b) electrodes made of PEDOT:PSS-coated PA 66—Round fibers. c) electrodes made of PEDOT:PSS-coated PA 6—Round fibers. d) electrodes made of PEDOT:PSS-coated PA 66—Ribbon fibers. e) electrodes made of PEDOT:PSS-coated PA 66—Trilobal fibers. n=5 electrodes per fiber material. Bars represent the mean and error bars are the standard error of the mean. ‘*’ symbol represents p<0.05.

In this work, electrocardiography chosen for on-body testing of electrodes due to its wide use in electrophysiological monitoring applications. ECG recordings were carried out by placing electrodes over the forearms as shown in FIG. 18a. As shown in FIGS. 18b-18f, all materials in every wash condition, except the 60× washed PES—Round electrodes showed no statistical difference between the R-peak-to-peak amplitudes recorded with textile electrodes and their gel equivalent.

FIGS. 18B-18F shows on-skin ECG measurements using unwashed, 30× washed, and 60× washed electrodes in accordance with illustrative embodiments, a) ECG recording methods. b) R-peak-to-peak amplitudes of gel electrodes vs. PEDOT:PSS-coated PES—Round electrodes. c) R-peak-to-peak amplitudes of gel electrodes vs. PEDOT:PSS-coated PA 66—Round electrodes. d) R-peak-to-peak amplitudes of gel electrodes vs. PEDOT:PSS-coated PA 6—Round electrodes. e) R-peak-to-peak amplitudes of gel electrodes vs. PEDOT:PSS-coated PA 66—Ribbon electrodes. f) R-peak-to-peak amplitudes of gel electrodes vs. PEDOT:PSS-coated PA 66-Trilobal electrodes. n=5 electrodes per fiber material. Bars represent the mean and error bars are the standard error of the mean. ‘*’ symbol represents p<0.05.

As described above, the teachings of the present disclosure can be generally applied to externally worn, ambulatory medical monitoring and/or treatment smart garments 110 suitable for defibrillation. Such garments 110 may include devices 100 that are not completely implanted within the patient's 102 body. The smart garment 110 can include, for example, ambulatory devices 100 that are capable of and designed for moving with the patient 102 as the patient goes about his or her daily routine in order to be capable of cardiac defibrillation. An example ambulatory smart garment 110 can be a wearable, such as a wearable cardioverter defibrillator (WCD), a wearable cardiac monitoring device, an in-hospital device such as an in-hospital wearable defibrillator, a short-term wearable cardiac monitoring and/or therapeutic device, mobile telemetry devices, and other similar wearable garments.

The wearable can be capable of continuous use by the patient. In some implementations, the continuous use can be substantially or nearly continuous in nature. That is, the wearable may be continuously used, except for sporadic periods during which the use temporarily ceases (e.g., while the patient bathes, while the patient is refit with a new and/or a different garment, while the battery is charged/changed, while the garment is laundered, etc.). Such substantially or nearly continuous use as described herein may nonetheless qualify as continuous use. For example, the wearable can be configured to be worn by a patient for as many as 24 hours a day without substantial interruption. In some implementations, the patient may remove the wearable for a short portion of the day (e.g., for half an hour to bathe).

Further, the wearable smart garment 110 can be configured as a long term or extended use. Such garments 110 can be configured to be used by the patient, continuously, on a daily basis, for an extended period of several days, weeks, months, or even years. In some examples, the wearable can be used by a patient, continuously, on a daily basis, for an extended period of at least one week. In some examples, the wearable can be used by a patient, continuously, on a daily basis, for an extended period of at least 30 days. In some examples, the wearable garment 110 can be used by a patient, continuously, on a daily basis, for an extended period of at least one month. In some examples, the wearable garment 110 can be used by a patient, continuously, on a daily basis, for an extended period of at least two months. In some examples, the wearable garment 110 can be used by a patient, continuously, on a daily basis, for an extended period of at least three months. In some examples, the wearable garment 110 can be used by the patient, continuously, on a daily basis, for an extended period of at least six months. In some examples, the wearable garment 110 can be used by a patient, continuously, on a daily basis, for an extended period of at least one year. In some implementations, the extended use can be uninterrupted until a physician or other caregiver provides specific instruction to the patient to stop use of the wearable garment 110.

Regardless of the extended period of wear, the use of the wearable garment 110 can include continuous or nearly continuous wear by the patient as described above. For example, the continuous use can include continuous wear or attachment of the wearable garment 110 to the patient, e.g., through one or more of the electrodes as described herein, during both periods of monitoring and periods when the device 100 may not be monitoring the patient but is otherwise still worn by or otherwise attached to the patient. The wearable garment 110 can be configured to continuously monitor the patient for cardiac-related information (e.g., electrocardiogram (ECG) information, including arrhythmia information, heart vibrations, etc.) and/or non-cardiac information (e.g., blood oxygen, the patient's temperature, glucose levels, tissue fluid levels, and/or lung vibrations). The wearable garment 110 can carry out its monitoring in periodic or aperiodic time intervals or times. For example, the monitoring during intervals or times can be triggered by a user action or another event.

As noted above, the wearable garment 110 can be configured to monitor other physiologic parameters of the patient in addition to cardiac related parameters. For example, the wearable can be configured to monitor, for example, lung vibrations (e.g., using microphones and/or accelerometers), breath vibrations, sleep related parameters (e.g., snoring, sleep apnea), tissue fluids (e.g., using radio-frequency transmitters and sensors), among others.

Other example wearable garments 110 include automated cardiac monitors and/or defibrillators for use in certain specialized conditions and/or environments such as in combat zones or within emergency vehicles. Such devices can be configured so that they can be used immediately (or substantially immediately) in a life-saving emergency. In some examples, the wearable garments 110 described herein can be pacing-enabled, e.g., capable of providing therapeutic pacing pulses to the patient.

In some implementations, the wearable ambulatory device may be operated in patient monitoring mode device where the treatment and/or therapy functions are removed/deactivated. For example, such a wearable ambulatory garment 110 can be configured to monitor one or more cardiac physiological parameters of a patient, e.g., for remotely monitoring and/or diagnosing a condition of the patient. For example, such cardiac physiological parameters may include a patient's ECG information, heart vibrations (e.g., using accelerometers or microphones), and other related cardiac information. In this regard, the wearable ambulatory garment 110 is configured to detect the patient's ECG through a plurality of cardiac sensing electrodes 112. Example cardiac conditions can include atrial fibrillation, bradycardia, tachycardia, atrio-ventricular block, Lown-Ganong-Levine syndrome, atrial flutter, sino-atrial node dysfunction, cerebral ischemia, syncope, atrial pause, and/or heart palpitations. When such an anomaly is detected, the monitor may automatically send data relating to the anomaly to a remote server. The remote server may be located within a 24-hour manned monitoring center, where the data is interpreted by qualified, cardiac-trained reviewers and/or caregivers, and feedback provided to the patient and/or a designated caregiver via detailed periodic or event-triggered reports. In certain cardiac event monitoring applications, the cardiac monitor is configured to allow the patient to manually press a button on the cardiac monitor (e.g., on the patient interface pod 140) to report a symptom. For example, a patient may report symptoms such as a skipped beat, shortness of breath, light headedness, racing heart rate, fatigue, fainting, chest discomfort, weakness, dizziness, and/or giddiness. The wearable ambulatory device can record predetermined physiologic parameters of the patient (e.g., ECG information) for a predetermined amount of time (e.g., 1-30 minutes before and 1-30 minutes after a reported symptom). The wearable ambulatory device can be configured to monitor physiologic parameters of the patient other than cardiac related parameters. For example, the wearable ambulatory device can be configured to monitor, for example, heart vibrations (e.g., using accelerometers or microphones), lung vibrations, breath vibrations, sleep related parameters (e.g., snoring, sleep apnea), tissue fluids, among others.

FIG. 19 schematically illustrates a sample component-level view of the controller 120. As shown in FIG. 19, the smart garment controller 120 can include a therapy delivery circuit 202, a data storage 204, a network interface 206, a user interface 208, at least one battery 210, a sensor interface 212, an alarm manager 214, and at least one processor 218. A patient monitoring smart garment 110 can include the controller 120 that includes like components as those described above, but does not include the therapy delivery circuit 202 (shown in dotted lines).

The therapy delivery circuit 202 can be coupled to one or more electrodes 220 configured to provide therapy to the patient (e.g., therapy electrodes 114 as described above). For example, the therapy delivery circuit 202 can include, or be operably connected to, circuitry components that are configured to generate and provide the therapeutic shock. The circuitry components can include, for example, resistors, capacitors, relays and/or switches, electrical bridges such as an h-bridge (e.g., including a plurality of insulated gate bipolar transistors or IGBTs), voltage and/or current measuring components, and other similar circuitry components arranged and connected such that the circuitry components work in concert with the therapy delivery circuit and under control of one or more processors (e.g., processor 218) to provide, for example, one or more pacing or defibrillation therapeutic pulses.

Pacing pulses can be used to treat cardiac arrhythmias such as bradycardia (e.g., less than 30 beats per minute) and tachycardia (e.g., more than 100 beats per minute) using, for example, fixed rate pacing, demand pacing, anti-tachycardia pacing, and the like. Defibrillation pulses can be used to treat ventricular tachycardia and/or ventricular fibrillation.

The capacitors can include a parallel-connected capacitor bank consisting of a plurality of capacitors (e.g., two, three, four or more capacitors). These capacitors can be switched into a series connection during discharge for a defibrillation pulse. For example, four capacitors of approximately 650 uF can be used. The capacitors can have between 350 to 500 volt surge rating and can be charged in approximately 15 to 30 seconds from a battery pack.

For example, each defibrillation pulse can deliver between 25 and 400 joules of energy (e.g., between 60 to 180 joules) of energy. In some implementations, the defibrillating pulse can be a biphasic truncated exponential waveform, whereby the signal can switch between a positive and a negative portion (e.g., charge directions). This type of waveform can be effective at defibrillating patients at lower energy levels when compared to other types of defibrillation pulses (e.g., such as monophasic pulses). For example, an amplitude and a width of the two phases of the energy waveform can be automatically adjusted to deliver a precise energy amount (e.g., 150 joules) regardless of the patient's body impedance. The therapy delivery circuit 202 can be configured to perform the switching and pulse delivery operations, e.g., under control of the processor 218. As the energy is delivered to the patient, the amount of energy being delivered can be tracked. For example, the amount of energy can be kept to a predetermined constant value even as the pulse waveform is dynamically controlled based on factors such as the patient's body impedance which the pulse is being delivered.

The data storage 204 can include one or more of non-transitory computer readable media, such as flash memory, solid state memory, magnetic memory, optical memory, cache memory, combinations thereof, and others. The data storage 204 can be configured to store executable instructions and data used for operation of the controller 120. In certain implementations, the data storage can include executable instructions that, when executed, are configured to cause the processor 218 to perform one or more functions.

In some examples, the network interface 206 can facilitate the communication of information between the controller 120 and one or more other devices or entities over a communications network. For example, where the controller 120 is included in an ambulatory (such as smart garment 110), the network interface 206 can be configured to communicate with a remote computing device such as a remote server or other similar computing device. The network interface 206 can include communications circuitry for transmitting data in accordance with a Bluetooth® wireless standard for exchanging such data over short distances to an intermediary device(s) (e.g., a base station, a “hotspot” device, a smartphone, a tablet, a portable computing device, and/or other devices in proximity of the wearable smart garment 110). The intermediary device(s) may in turn communicate the data to a remote server over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with a remote server over a Wi-Fi™ communications link based on the IEEE 802.11 standard.

In certain implementations, the user interface 208 can include one or more physical interface devices such as input devices, output devices, and combination input/output devices and a software stack configured to drive operation of the devices. These user interface elements may render visual, audio, and/or tactile content. Thus, the user interface 208 may receive input or provide output, thereby enabling a user to interact with the controller 120.

The controller 120 can also include at least one battery 210 configured to provide power to one or more components integrated in the controller 120. The battery 210 can include a rechargeable multi-cell battery pack. In one example implementation, the battery 210 can include three or more 2200 mAh lithium-ion cells that provide electrical power to the other device components within the controller 120. For example, the battery 210 can provide its power output in a range of between 20 mA to 1000 mA (e.g., 40 mA) output and can support 24 hours, 48 hours, 72 hours, or more, of runtime between charges. In certain implementations, the battery capacity, runtime, and type (e.g., lithium ion, nickel-cadmium, or nickel-metal hydride) can be changed to best fit the specific application of the controller 120.

The sensor interface 212 can be coupled to one or more sensors configured to monitor one or more physiological parameters of the patient. As shown, the sensors may be coupled to the controller 120 via a wired or wireless connection. The sensors can include one or more electrocardiogram (ECG) electrodes 112 (e.g., similar to sensing electrodes 112 as described above), heart vibrations sensors 224, and tissue fluid monitors 226 (e.g., based on ultra-wide band radiofrequency devices).

The ECG electrodes 112 can monitor a patient's ECG information. For example, the ECG electrodes 112 can be galvanic (e.g., conductive) and/or capacitive electrodes configured to measure changes in a patient's electrophysiology to measure the patient's ECG information. The ECG electrodes 112 can transmit information descriptive of the ECG signals to the sensor interface 212 for subsequent analysis.

The heart vibrations sensors 224 can detect a patient's heart vibration information. For example, the heart vibrations sensors 224 can be configured to detect heart vibration values including any one or all of S1, S2, S3, and S4. From these heart vibration values, certain heart vibration metrics may be calculated, including any one or more of electromechanical activation time (EMAT), percentage of EMAT (% EMAT), systolic dysfunction index (SDI), and left ventricular systolic time (LVST). The heart vibrations sensors 224 can include an acoustic sensor configured to detect vibrations from a subject's cardiac system and provide an output signal responsive to the detected heart vibrations. The heart vibrations sensors 224 can also include a multi-channel accelerometer, for example, a three channel accelerometer configured to sense movement in each of three orthogonal axes such that patient movement/body position can be detected and correlated to detected heart vibrations information. The heart vibrations sensors 224 can transmit information descriptive of the heart vibrations information to the sensor interface 212 for subsequent analysis.

The tissue fluid monitors 226 can use radio frequency (RF) based techniques to assess fluid levels and accumulation in a patient's body tissue. For example, the tissue fluid monitors 226 can be configured to measure fluid content in the lungs, typically for diagnosis and follow-up of pulmonary edema or lung congestion in heart failure patients. The tissue fluid monitors 226 can include one or more antennas configured to direct RF waves through a patient's tissue and measure output RF signals in response to the waves that have passed through the tissue. In certain implementations, the output RF signals include parameters indicative of a fluid level in the patient's tissue. The tissue fluid monitors 226 can transmit information descriptive of the tissue fluid levels to the sensor interface 212 for subsequent analysis.

The sensor interface 212 can be coupled to any one or combination of sensing electrodes/other sensors to receive other patient data indicative of patient 102 parameters. Once data from the sensors has been received by the sensor interface 212, the data can be directed by the processor 218 to an appropriate component within the controller 120. For example, if heart data is collected by heart vibrations sensor 224 and transmitted to the sensor interface 212, the sensor interface 212 can transmit the data to the processor 218 which, in turn, relays the data to a cardiac event detector. The cardiac event data can also be stored on the data storage 204.

In certain implementations, the alarm manager 214 can be configured to manage alarm profiles and notify one or more intended recipients of events specified within the alarm profiles as being of interest to the intended recipients. These intended recipients can include external entities such as users (patients, physicians, and monitoring personnel) as well as computer systems (monitoring systems or emergency response systems). The alarm manager 214 can be implemented using hardware or a combination of hardware and software. For instance, in some examples, the alarm manager 214 can be implemented as a software component that is stored within the data storage 204 and executed by the processor 218. In this example, the instructions included in the alarm manager 214 can cause the processor 218 to configure alarm profiles and notify intended recipients using the alarm profiles. In other examples, alarm manager 214 can be an application-specific integrated circuit (ASIC) that is coupled to the processor 218 and configured to manage alarm profiles and notify intended recipients using alarms specified within the alarm profiles. Thus, examples of alarm manager 214 are not limited to a particular hardware or software implementation.

In some implementations, the processor 218 includes one or more processors (or one or more processor cores) that each are configured to perform a series of instructions that result in manipulated data and/or control the operation of the other components of the controller 120. In some implementations, when executing a specific process (e.g., cardiac monitoring), the processor 218 can be configured to make specific logic-based determinations based on input data received, and be further configured to provide one or more outputs that can be used to control or otherwise inform subsequent processing to be carried out by the processor 218 and/or other processors or circuitry with which processor 218 is communicatively coupled. Thus, the processor 218 reacts to specific input stimulus in a specific way and generates a corresponding output based on that input stimulus. In some example cases, the processor 218 can proceed through a sequence of logical transitions in which various internal register states and/or other bit cell states internal or external to the processor 218 may be set to logic high or logic low. As referred to herein, the processor 218 can be configured to execute a function where software is stored in a data store coupled to the processor 218, the software being configured to cause the processor 218 to proceed through a sequence of various logic decisions that result in the function being executed. The various components that are described herein as being executable by the processor 218 can be implemented in various forms of specialized hardware, software, or a combination thereof. For example, the processor can be a digital signal processor (DSP) such as a 24-bit DSP processor. The processor can be a multi-core processor, e.g., having two or more processing cores. The processor can be an Advanced RISC Machine (ARM) processor such as a 32-bit ARM processor. The processor can execute an embedded operating system, and include services provided by the operating system that can be used for file system manipulation, display & audio generation, basic networking, firewalling, data encryption and communications.

Although the subject matter contained herein has been described in detail for the purpose of illustration, it is to be understood that such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

Other examples are within the scope and spirit of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

1. A non-invasive, wearable, ambulatory device capable of cardiac defibrillation, the device comprising:
a smart garment configured to be worn about a torso of a patient;
a plurality of therapeutic electrodes configured to be removably attached to the garment;
a plurality of polymer-based ECG sensing electrodes configured to provide ECG signals based on skin electrical activity of the patient wearing the smart garment, wherein one or more of the plurality of polymer-based ECG sensing electrodes comprises:
a plurality of individually conductive polymer coated fibers, wherein each of the plurality of the individually conductive polymer coated fibers comprises a base fiber treated with a conductive polymer fluid disposed along the base fiber, the base fiber being a single fiber and/or multifiber, and
a conductive polymer coated fiber assembly comprising the plurality of the individually conductive polymer coated fibers arranged in a predetermined configuration; and
a controller in electrical communication with the plurality of therapeutic electrodes and the plurality of polymer-based ECG sensing electrodes, the controller configured to:
receive the ECG signals;
determine at least one arrhythmia episode occurring in the patient based on the received ECG signals; and
causing a defibrillation shock to be delivered to the patient via the plurality of therapeutic electrodes as a function of determining the occurrence of the at least one arrhythmia episode.

2. (canceled)

3. The device of claim 1, wherein the base fiber is a non-conductive fiber.

4. The device of claim 1, wherein a stretchable fabric portion of the smart garment at least partially surrounds the polymer-based ECG sensing electrodes.

5. (canceled)

6. The device of claim 1, wherein the conductive polymer fluid comprises poly(3,4-ethylenedioxythiophene) polystyrene sulfonate (PEDOT:PSS).

7. (canceled)

8. The device of claim 1, wherein one or more of the polymer-based ECG sensing electrodes are configured to be removably attached to the smart garment.

9. (canceled)

10. The device of claim 1, wherein the conductive polymer fluid has a surface tension of between about 30 mN/m and about 45 mN/m, or about 35 mN/m and about 40 mN/m, or about 39 mN/m.

11. (canceled)

12. The device of claim 1, further comprising a conductive polymer fiber interconnect configured to electrically couple two of the plurality of polymer-based ECG sensing electrodes.

13. The device of claim 12, wherein the conductive polymer fiber interconnect is formed by assembling the plurality of the individually conductive polymer coated fibers in a longitudinal pattern between two of the plurality of polymer-based ECG sensing electrodes.

14-18. (canceled)

19. The device of claim 1, wherein a surface of the base fiber is pre-treated with plasma prior to applying the conductive fluid.

20. (canceled)

21. (canceled)

22. The device of claim 1, wherein the conductive polymer coated fiber assembly is assembled by weaving the plurality of individually conductive polymer coated fibers.

23. The device of claim 1, wherein the conductive polymer coated fiber assembly is assembled by knitting the plurality of individually conductive polymer coated fibers.

24. (canceled)

25. The device of claim 1, wherein the polymer-based ECG sensing electrodes each have a signal-to-noise ratio of between 2.5 and 30.1 for the received ECG signals.

26. The device of claim 1, wherein the polymer-based ECG sensing electrodes each have a skin-electrode impedance value of between 65 kOhms and 105 kOhms at 100 Hz.

27. The device of claim 26, wherein the polymer-based ECG sensing electrodes resistance changes less than a predetermined 50% of a baseline impedance value from about 10 Hz to about 500 Hz after 30 wash cycles.

28-44. (canceled)

45. A method of making a smart garment for cardiac health monitoring comprising:
individually coating each of a plurality of single fibers and/or multifibers with a conductive polymer coating fluid to form a plurality of conductive fabric fibers;
assembling the plurality of conductive fabric fibers to form an electrically conductive fabric portion of a smart garment, the electrically conductive fabric portion forming an ECG electrode configured to sense ECG signals from a patient; and
forming a stretchable fabric portion of the smart garment at least partially surrounding the electrically conductive fabric portion.

46. The method of claim 45, wherein the stretchable fabric portion has a first yield strain value, and the electrically conductive fabric portion has a second yield strain value that is less than the first yield strain value.

47. The method of claim 45, wherein assembling comprises knitting, weaving, or embroidering.

48. The method of claim 45, wherein the ECG electrode is knitted using a Stoll CMS-ADF flatbed knitting machine.

49. The method of claim 45, further comprising curing the plurality of conductive fabric fibers before assembling the plurality of conductive fabric fibers.

50. The method of claim 45, wherein curing comprises continuously moving the fibers through an oven.

51. The method of claim 45, wherein curing comprises heating the fibers at a temperature of between about 190 C and 220 C.

52. The method of claim 45, wherein a coating speed is between 10 rpm and 40 rpm.

53. The method of claim 45, wherein coating is deposited on the fiber at a rate of between 50 uL/min and 150 uL/min.

54. The method of claim 45, wherein the linear density of the coating is between 20 uL/m and 35 uL/m.

55-70. (canceled)
